(19) United States
(12) Patent Application Publication
(10) Pub. No.: US 2011/0059864 A1
(43) Pub. Date: Mar. 10, 2011

(54) Sequence Determination By Use Of Opposing Forces

(75) Inventors: Javier Farinas, Los Altos, CA (US); Andrea Chow, Los Altos, CA (US); John Wallace Parce, Palo Alto, CA (US)

(73) Assignee: CAERUS MOLECULAR DIAGNOSTICS INCORPORATED, Los Altos, CA (US)

(21) Appl. No.: 12/877,103

(22) Filed: Sep. 7, 2010

Related U.S. Application Data
(60) Provisional application No. 61/240,304, filed on Sep. 7, 2009.

(51) Int. Cl. C40B 30/10; C12M 1/34

(52) U.S. Cl. 506/12; 435/287.1

(57) ABSTRACT

The present teachings relate to systems, methods, and the like, for analyzing biological polymers, by use of opposing forces. Among other things, the present teachings can be used to determine sequence information, such as in genetic sequencing and genotyping applications. Various embodiments are described for efficient, high throughput sequencing of nucleic-acid molecules, such as DNA. Various embodiments are described wherein nucleic-acid sequence information is determined without the need or use of extrinsic labels. As well various embodiments of methods, systems, and the like, are described, which can provide long and accurate read lengths for low-cost nucleic acid sequencing.

### CROSS-REFERENCE TO RELATED APPLICATIONS

This application claims a priority benefit under 35 U.S.C. §119(e) from U.S. Patent Application No. 61/240,304, filed Sep. 7, 2009, which is incorporated herein by reference in its entirety.

### FIELD

The present teachings relate to the field of genetic analysis; and, more particularly, to sequence determination of biological polymers, such as nucleic-acid molecules.

### BACKGROUND

Rapid, accurate, and inexpensive characterization of biological polymers, such as genetic material (e.g., DNA), has become increasingly important. For example, sequencing the equivalent of an entire human genome for $1,000 has been announced as a goal for the genetics community. That is generally considered to be the threshold cost allowing for widespread adoption and use of whole genome sequencing. While the cost to sequence a mammalian genome has dropped dramatically over the past decade or so, routine sequencing of organisms for basic research and human samples for translational research and individualized health care is still limited by high costs. It is estimated that another two orders of magnitude decrease in cost is needed in order to achieve the goal of $1,000 per genome.

In the past few years, a new generation of DNA sequencing platforms has emerged, based on fundamentally different methodologies as compared to the technologies that dominated during the preceding decades (particularly, Sanger-based sequencing methodologies). These relatively recent, so-called “next-generation” (or “next-gen”) sequencing technologies may create a new set of research opportunities for the coming decade. However, despite technological advancements and substantial financial investment, no clear path has emerged to achieve the $1,000 genome.

The fundamental challenge is to maintain high standards of accuracy and completeness of sequencing, while reducing cost in three key areas: sample preparation, sequence detection, and genome data assembly. Several attributes are generally desirable in order for a technology to significantly lower sequencing cost; which are: simplified sample preparation, low reagent cost, low instrument amortization cost, long read length, and high accuracy. The currently available, or otherwise known, sequencing systems are inadequate in these regards.

A sequencing system that is lower in cost than the available or known systems, yet which exhibits high throughput and acceptable accuracy is desired, as such as system would be a substantial advancement towards the goal of a $1,000 genome.

### SUMMARY OF VARIOUS EMBODIMENTS

An exemplary and non-limiting summary of various embodiments is set forth next. The following various embodiments and examples are offered for illustrative purposes only and are not intended to limit the scope of the present teachings in any way.

In various aspects of the present teachings, opposed forces (also referred to as opposing forces) can be embodied in a variety of methods, systems, and the like, for analyzing one or more biological polymers. In a variety of embodiments, for example, opposed forces facilitate identifying or otherwise characterizing one or more monomer units of a biological polymer. For example, opposed forces can facilitate analysis of nucleic-acid molecules, such as RNA, DNA and the like (e.g., in sequencing, genotyping, etc.). As well, systems and methods as taught herein can find use in a variety of fields and applications, such as basic biological research, diagnostics, biotechnology, forensics, and personalized medicine.

Generally, according to various aspects of the teachings herein, two or more opposing forces act upon a solid support (e.g., a particle, bead, and the like), linked to a biological polymer. The particle tends to a first state characterized for example by a location and/or velocity due to the balance of forces. An event alters the biological polymer, consequently causing a change to a detectable property of the particle. Detection of the latter provides structural information about the biological polymer.

The present teachings, in a variety of embodiments, relate to methods, systems, and the like, for analyzing nucleic-acid molecules. A number of such embodiments provide systems and methods for (i) generating a set of particles which are associated with a clonal set of nucleic acids; (ii) applying an electric force and opposing forces to the particles in an aqueous medium; (iii) measuring a parameter which is a function of the charge on the particle; (iv) changing the number of nucleotides on the nucleic acid; (v) repeating steps (iii) and (iv); and (vi) analyzing resulting changes in the parameter to determine at least one characteristic of the nucleic-acid molecules. In some embodiments, the characteristic of step (vi) includes sequence information.

According to various embodiments, the particles can comprise, e.g., beads, liposomes, micelles, lipid coated beads, polymer coated beads, oil droplets, detergent or lipid or polymer coated oil droplets, polymers, or quantum dots, or any combination thereof. In some embodiments, molecules analyzed by the methods herein include nucleic-acid molecules, such as RNA, or DNA, or the like. Various embodiments of the methods here contemplate use of one or more opposing forces selected from the following group: electrical force, optical force, magnetic force, entropic force, hydrodynamic force, gravitational force, centrifugal force, mechanical force, dielectrophoretic force, and any combination thereof. In some embodiments, an opposing force can be generated by a means of trapping. Such means can include, e.g., optical traps, magnetic traps, dielectrophoretic traps, and the like. Certain embodiments contemplate generation of one or more opposing forces generated by mechanical and/or micromechanical means. In certain embodiments, one or more opposing forces are generated by way of a cantilever and/or a centrifuge. Various embodiments of the present teachings employ a surface attached molecule.

In accordance with some embodiments of the methods herein, the parameter is measured optically. In a variety of embodiments, the parameter which is measured can be position, velocity, or acceleration of a particle, or the net force on a particle, or a combination of the foregoing. In a number of embodiments, a position of one or more particles can be measured with a fast quadrant diode detector. Various embodiments contemplate employing a CCD or CMOS device, or the like, (e.g., a fast CCD or CMOS camera or similar array detector).

According to a number of embodiments, one or more opposing forces can be applied, for example, in at least one of the following ways: as an impulse, a periodic, and/or a steady manner.

Various embodiments herein contemplate changing the number of nucleotides on a nucleic-acid molecule by synthesizing nucleotides (e.g., any suitable type of sequencing by synthesis (SBS) approach). According to a number of embodiments, the number of nucleotides can be changed by cleaving nucleotides (e.g., employing one or more enzymes including a nuclease activity, e.g., exonuclease activity, endonuclease activity; and/or by chemical cleavage). In various embodiments, the number of nucleotides is changed by hybridizing and/or ligating nucleotides. A variety of embodiments contemplate adding nucleotides sequentially by a polymerase by adding solutions containing one or predominantly one nucleotide at a time. In some embodiments, nucleotides added to the nucleic acid can be distinguished by charge labels. With some embodiments nucleotides cleaved off the nucleic acid are distinguished by charge labels.

In a variety of embodiments, according to the teachings herein, the number of particles measured is 1, more than 1, more than 100, more than 10,000, more than 1,000,000, more than 10,000,000. In various embodiments, the number of nucleic acids on the particle is 1, more than 1, more than 100, more than 10,000, more than 1,000,000.

In accordance with various embodiments, the charge on the particles without the bound nucleic acid is made close to neutral.

In some embodiments, the change in charge and the measurement are done concurrently.

Other aspects of the present teachings are embodied in a variety of systems and methods adapted, for example, to sequence nucleic acids. Various embodiments include, among other things: (i) generating a set of beads of which each has 1 or more (e.g., in some embodiments, e.g., within a range of from 1 to about 1,000,000; in some other embodiments, more than 1,000,000) copies of nucleic acids; (ii) applying an AC electric field and an opposing force from an array of optical traps to the particles in an aqueous medium; (iii) measuring the displacement of the bead from the center of the trap; (iv) sequentially bathing the beads in polymerization solutions containing polymerases and one of the 4 nucleotides (A, G, C or T/U); (v) Repeating steps (iii) and (iv); and (v) analyzing the resulting changes in bead position or Zeta potential to define the nucleic acid sequences.

Further aspects of the present teachings relate to systems and methods for sequencing nucleic acids. Various embodiments, relating to such further aspects, include, for example: (i) generating a set of beads of which each has 1 or more (e.g., in some embodiments, e.g., within a range of from 1 to about 1,000,000; in some other embodiments, more than 1,000,000) copies of nucleic acids; (ii) applying an AC electric field and an opposing force from an array of magnetic traps to the particles in an aqueous medium; (iii) measuring the displacement of the bead from the center of the trap; (iv) sequentially bathing the beads in polymerization solutions containing polymerases and one of the 4 nucleotides (A, G, C or T/U); (v) repeating steps (iii) and (iv); and (vi) analyzing the resulting changes in bead position or Zeta potential to define the nucleic acid sequences.

Additionally, a number of embodiments, according to the teachings here, provide a system and method for sequencing nucleic acids. Various embodiments, in these regards, include: (i) generating a set of beads of which each has 1 or more (e.g., in some embodiments, e.g., within a range of from 1 to about 1,000,000; in some other embodiments, more than 1,000,000) copies of nucleic acids; (ii) applying an AC electric field and an opposing force by attaching the beads to a surface by a polymer in an aqueous medium; (iii) measuring the displacement of the beads from the central position; (iv) sequentially bathing the beads in polymerization solutions containing polymerases and one of the 4 nucleotides (A, G, C or T/U); (v) repeating steps (iii) and (iv); and (iv) analyzing the resulting changes in bead position or Zeta potential to define the nucleic acid sequences.

### BRIEF DESCRIPTION OF FIGURES

These and other embodiments of the disclosure will be discussed with reference to the following non-limiting and exemplary illustrations, in which like elements are numbered similarly, and where:

FIG. 1 is a schematic representation of a particle experiencing an electrical force and an opposing optical force from a tightly focused laser beam (optical trap), according to various embodiments of the present teachings.

FIG. 2 shows, in schematic form, (i) steps for sequencing, using the optical trap of FIG. 1, wherein a change is made to the number of nucleotides on a nucleic-acid molecule bound to a particle; and (ii) resulting changes in position (i.e., an observable parameter) of the particle within opposing fields of the optical trap; according to various embodiments of the present teachings.

FIG. 3 (A) depicts, in schematic fashion, plural forces acting on a particle; particularly, (i) an electrical force, and (ii) opposed forces comprised of (a) a hydrodynamic force and (b) a restoring “spring-like” force; according to various embodiments of the present teachings.

FIG. 3 (B) is a schematic representation of a tethered bead, having a DNA template attached to it for sequencing; charges are shown on nucleic acid chains; according to various embodiments of the present teachings.

FIG. 4 depicts, in schematic fashion, one round of sequencing-by-synthesis, using the system of FIG. 3, according various embodiments of the present teachings, wherein opposing forces act on a bead associated with a nucleic-acid molecule of interest, with the forces including an electrical force and two opposing forces: a hydrodynamic force and a spring-like restoring force. Steps are depicted illustrating a change in bead position upon addition of a nucleotide to the nucleic-acid carried by the bead; according to various embodiments of the present teachings.

FIG. 5(A) graphically illustrates the predicted dependence of Zeta potential on the number of added nucleotides for 0.5 micrometer (sometimes indicated as um or μm) beads having 500 templates per bead; according to various embodiments of the present teachings.

FIG. 5(B) graphically illustrates the predicted sensitivity as a function of the number of added nucleotides; and, also, the predicted sensitivity required for each of 99% accuracy and 99.9% accuracy; according to various embodiments of the present teachings.

FIG. 6 graphically illustrates the predicted sensitivity as a function of the number of added nucleotides; and, also, the sensitivity required for each of 99% and 99.9% accuracy for 1 μm beads over an observation period of 7 seconds; according to various embodiments of the present teachings.

FIG. 7 graphically illustrates the predicted sensitivity as a function of the number of added nucleotides; and, also, sensitivity required for each of 99% accuracy and 99.9% accuracy for 1 μm beads over an observation period of seven seconds; according to various embodiments of the present teachings.

FIG. 8 schematically depicts a chip-type flow cell providing for rapid nucleotide exchange; according to various embodiments of the present teachings.

FIG. 9 schematically depicts an optical system, including dark-field illumination and a quadrant photodiode, adapted to measure the Zeta potential of a tethered microbead; according to various embodiments of the present teachings.

FIG. 10 schematically depicts a flow cell, which comprises a fluidic network including 4 separate reservoirs for introducing the 4 dNTP's independently into a bead chamber area containing captured beads, for sequencing experiments, in accordance with various embodiments of the present teachings.

FIGS. 11(A), (B), (C), and (D) illustrate, in schematic fashion, various schemes for mapping of one or more beads to pixels of an imaging device, as a means to measure bead position; according to various embodiments of the present teachings.

### DESCRIPTION

### Introduction

The present teachings provide, among other things, methods, systems, and the like, for analyzing one or more biological polymers of interest. Opposing forces can be used, as further described herein, in determining sequence information of genetic materials. As will become apparent, the present teachings are well suited for use in the field of genetic sequencing; and, in particular, with regard to recent and ongoing efforts aimed at revolutionizing sequencing via non-Sanger-based approaches (e.g., “next-generation sequencing” (NGS); including “second-generation’; as well as “third-generation,” or, “single-molecule sequencing” (SMS), approaches).

In general, various aspects of the present teachings provide for analysis of one or more nucleic-acid molecules, such as DNA, by use of opposing forces, such as for example an electrical force and at least one other source. As will be appreciated by those skilled in the art, opposing forces can be embodied in a variety of systems and methods, such as taught herein.

For example, in some embodiments, at least one particle, bead, or the like, can be tethered to a support. At least one nucleic acid template (e.g., DNA or RNA) can be bound to the particle. Sequence information about the nucleic acid can be obtained by a change in charge that can be measured as nucleotides are added to, or removed from, or hybridized to, the attached nucleic acid. Opposing forces, such as an electrical force, which has a strong dependence on the number of nucleotides in the nucleic acid template, and hydrodynamic and restoring, spring-like forces which have a weak or no dependence on the number of nucleotides in the nucleic acid template, can result in detectable motion of the particle, which is a function of the length of DNA attached to it. Simultaneous detection of a large quantity of template-bearing particles, e.g., an array of particles, under conditions appropriate for nucleic acid elongation (e.g., via a sequencing-by-synthesis, or SBS, scheme), can facilitate high-throughput sequencing. By way of opposed-force sequencing, in accordance with the present teachings, long and accurate read lengths can be achieved.

Opposed-force sequencing can be carried out, in various embodiments, without the use or need of labels (e.g., fluorescent labels). For example, in various systems and methods, as taught and variously embodied herein, there is an absence of any fluorescent labels, at least insofar as reagents are concerned (e.g., free of fluorescently labeled nucleotides, whether covalently labeled or otherwise). Being label-free, opposed-force sequencing systems can avoid costly reagents commonly used with other known systems. As well, with label-free sequencing, systems for detecting particle motion can be relatively simple, thereby avoiding most, if not all, of the complex, expensive detection assemblies often found in other sequencing systems. In this regard, for example, various embodiments herein employ simple dark-field optics.

Long read lengths achievable using opposed-force sequencing, as taught herein, can simplify the task of genome assembly, as compared to relatively short reads often encountered with other non-Sanger-based systems. It is noted that short read lengths typically cause sequence assembly to be very resource intensive.

Opposed-force sequencing, as taught in various embodiments, can be employed for sequencing a single template (i.e., single-molecule sequencing, or SMS), or used in various amplification-based schemes. In various embodiments of single-molecule sequencing, a single DNA template can be sequenced by monitoring changes in the intrinsic charge of a growing DNA chain. By utilizing a single-template approach, the need for amplification (e.g., emulsion PCR or bridge PCR) can be avoided, thereby simplifying sample preparation.

Further discussion, description of various embodiments, and illustrative examples are provided herein.

### DEFINITIONS

In considering the embodiments below, as well as elsewhere herein, the following definitions should be taken into account. As well, consideration should be given to what those skilled in the art would understand, within the overall context of the present teachings.

The singular terms “a”, “an,” and “the” include plural referents unless the content clearly indicates otherwise. Thus, for example, reference to “a nucleic acid” includes two or more such nucleic acids (e.g., as in a mixture), and the like.

The terms “hybridize” and “hybridization” refer to the formation of complexes between nucleotide sequences that are sufficiently complementary to form complexes, e.g., via Watson-Crick base pairing.

The term “cantilever” refers to a beam supported only on one end. For example, such cantilevers can be made, e.g., from micropipettes, from microfabrication of Si or polymers or from protein structures such as actin filaments.

The term “optical trap” refers to the use of light to apply forces to particles. Forces can be applied so that the position of the particle tends to a location in an aqueous medium due to a restoring force. For example, traps can be made by tightly focusing a laser beam or by opposing fiber optics.

The term “magnetic trap” refers to the use of magnetic field gradients to apply forces to one or more supports, such as one or more particles. Forces can be applied so that the position of a particle tends to a location in an aqueous medium due to a restoring force.

The term “polymer” refers to a molecule (often large) comprised of repeating structural units, or monomers, connected by covalent bonds. Examples of polymers include, without limitation, DNA, RNA, proteins, oligosaccharides, polyethylene, polyethylene oxide, etc.

A “polymer replicating catalyst,” “polymerizing agent” or “polymerizing catalyst,” is an agent that can catalytically assemble monomers into a polymer in a template dependent fashion; that is, in a manner that uses the polymer molecule originally provided as a template for reproducing that molecule from at least one or more suitable monomers. Such agents include, but are not limited to, catalytic proteins, such as enzymes, including nucleotide polymerases, e.g., DNA polymerases, RNA polymerases, tRNA and ribosomes.

The term “entropic force” refers to a force whose properties are primarily determined not by the character of a particular underlying microscopic force (such as, for example, electromagnetism), but by the whole system's statistical tendency to increase its entropy. A non-limiting example of an entropic force is the elasticity of a freely-jointed polymer molecule.

The terms “signal”, “parameter” and “signal parameter” as refer to a property of a support, such as a particle; such as position, velocity, acceleration or net force on the particle.

Generally, deoxyribonucleic acid (DNA) comprises polymeric strands of nitrogenous bases. The bases typically include purines (adenine and guanine, abbreviated as A and G, respectively) and pyrimidines (cytosine and thymine, abbreviated as C and T, respectively). Typically (e.g., under natural conditions), the strands configure as a double helix, with bases to the center (like rungs on a ladder) and sugar-phosphate units along the sides of the helix (like the sides of a twisted ladder). The strands tend towards complementarity (i.e., A pairing with T, and C pairing with G).

It is well established that nucleic-acid polymers generally comprise a sequence of linked-together nucleotide units or monomers (e.g., deoxyribonucleotide (DNA), ribonucleotide (RNA), and/or modifications or analogs thereof), and include a phosphate backbone that confers a net negative charge on the molecule. Such nucleic-acid polymers are often referred to in the relevant arts as “oligonucleotides” or “polynucleotides.” As is well known, for example in the field of electrophoresis, nucleic acids are polyelectrolytes whose physical properties and chemical reactivity are affected by the pH and ionic strength of a solution. In addition to the ionizable phosphodiester internucleotide bonds, the bases can be ionized or protonated depending upon the pH. The pK values of the nucleoside and sugar-phosphate backbone components of deoxyribo- and ribonucleic acids are well characterized. The pKa of the phosphate group, i.e., the measure of how readily that group will give up a hydrogen cation proton, is near 1. Thus, under most ionic condition, including physiological pH, the backbone will contain a single negative charge for each nucleotide unit, or two negative charges for a Watson-Crick pair of nucleotides in a double strand. (Zwolak, Michael and Di Ventra, Massimiliano (2008), Colloquium: Physical approaches to DNA sequencing and detection. Reviews of Modern Physics, 80 (1). pp. 141-165. ISSN 0034-6861.) Under physiological conditions of pH, the phosphodiester bonds are ionized, whereas the bases are in a neutral form, and thus the nucleic acid has an overall net negative charge.

Because each nucleotide is ionized, the charge-to-mass ratio of two different nucleic acid molecules will very closely agree. (Recombinant DNA principles and methodologies, edited by James J. Greene, Venigalla B. Rao. Published 1998, Marcel Dekker, New York). Under influence of an electric force, negatively charged nucleic acid molecules tend towards a positive pole in an electrical field.

As a brief aside, it should be noted that the terms “oligonucleotides” and “polynucleotides,” among others (e.g., “nucleic acid” and “nucleic-acid molecule”) often encountered in the relevant arts, are sometimes used to convey features or structural information (albeit, typically in a generalized, high-level fashion). For example, “oligonucleotide” is sometimes intended and understood to indicate generally short sequences, and “polynucleotide” is sometimes intended and understood to indicate generally long sequences (in other words, “oligonucleotides” tend be short, relative to “polynucleotides”). Notwithstanding the foregoing, it should further be noted that on many occasions such terms are used by those in the art in an imprecise fashion; without thought or intent of conveying features or structural information. In this regard, it is not uncommon for such terms to be used interchangeably. From knowledge and experience, persons regularly working and/or skilled in the relevant arts will appreciate the varied usage of these terms.

From knowledge, experience, and consideration of the overall context in which the terms as discussed above are used, those skilled in the art will appreciate the meaning of such terms herein. For example, in various contexts, the terms “polynucleotide,” “oligonucleotide,” and “nucleic-acid molecule” will be understood to include polymeric forms of nucleotides of any length (i.e., number of sequential bases), either ribonucleotide or deoxyribonucleotide.

Unless clear otherwise, terms herein such as “polynucleotide, “nucleic acid,” “oligonucleotide,” and “nucleic-acid molecule,” refer to the primary structure of the relevant molecules. Thus, for example, such terms include triple-, double-, and single-stranded RNA, as well as triple-, double-, and single-stranded DNA. As well, they include modified forms, such as by methylation and/or by capping, and unmodified forms. Generally, there is not intended any distinction in length between the terms “polynucleotide,” “oligonucleotide,” “nucleic acid,” and “nucleic acid molecule,” and these terms may be used interchangeably herein. These terms can include, for example, double- and single-stranded DNA, as well as double- and single-stranded RNA, DNA:RNA hybrids, and also various modifications, for example, labels (as known in the art), methylation, “caps,” substitution of one or more of the naturally occurring nucleotides with an analog; and modified nucleic acids, such as locked nucleic acids; as well as unmodified forms of polynucleotide or oligonucleotide.

As used in connection with an analysis of polymeric molecules associated with a support, plural forces are considered herein to be “opposing,” or “opposed to,” each other when at least two of the plural forces acting on the support include vector components that are pointing in opposite directions. As well, for purposes herein, at least one force of the plural forces is typically characterized by a dependence on (or, in other words, is a function of) the charge, and changes made to the charge, of the charged polymeric molecule(s) analyzed. In a variety of embodiments, such dependence is a strong one, and at least one other force of the plural forces is characterized by only a weak or no dependence on the charge of the charged polymeric molecule(s) analyzed. Still further, in various embodiments, the latter force is additionally characterized by a restoring, spring-like quality. The forces can include, but are not limited to, body and surface forces acting on the monomer units of the polymeric molecule(s) analyzed, and/or on the support. Exemplary forces comprise, without limitation, electrical, dielectrophoretic, mechanical, hydrodynamic, entropic, magnetic, optical, etc. The foregoing is provided for illustrative purposes. It is noted that, among other things, the choice of forces, their directions of application, and the magnitude of their strength can vary.

### Various Embodiments

Reference will now be made to various non-limiting embodiments, various examples of which are illustrated in the accompanying drawings.

At the outset, it is noted that aspects of the present teachings relate to systems and methods for sequence determination using opposed-forces, comprising application of a force that depends directly on the number of monomers of a polymeric molecule of interest, and further wherein such number of monomers can be changed (under appropriate conditions) by a suitable event or means.

In addition, it should be noted that “length” and/or “size” or “charge” are sometimes used as an indicator of (i.e., in a sense, as a proxy for) the number of monomer units that comprise a polymeric molecule. Thus, a change (i) in “length”, “size” or “charge” of a polymeric molecule can be considered to equate with a change (ii) in the number of monomer units that comprise it. Further, a change to a polymeric molecule articulated herein as comprising either, or both, a change in (i) length or size, or (ii) charge, can be considered as comprising a process step or means for effecting a change in the number of monomer units of the molecule. In many embodiments described herein, it is more natural to consider the forces acting on the polymer of interest due to the overall or net charge of the polymer. Thus, for example, with regard to an analysis of a biological polymer associated with a support element, at least one of the opposing forces applied typically depends directly on the overall charge of the polymer (and, correspondingly, on the length, size, and number of monomer units comprising the polymer). For example, with regard to a charged nucleic-acid molecule, at least one of the applied forces is typically characterized by a dependence on such charge. The charge, in turn, is a function of the number of nucleic-acid monomer units comprising the nucleic-acid molecule. Upon, for example, adding or removing nucleic-acid monomer units under appropriate conditions (e.g., within an appropriate pH range), the overall charge of the nucleic-acid molecule can be changed. It should also be noted that the foregoing is not intended to be limiting with regard to process steps or means for effecting a change to the overall number of monomers of the molecule.

As further described herein, various embodiments provide, for example, methods, systems, sub-systems, apparatus, components, processes, assays, reagents, and the like, for analyzing one or more biological molecules, including various biological polymers, such as one or more polymeric strands comprised of nucleic acids (e.g., nucleic-acid molecules, polynucleotides, oligonucleotides, DNA, RNA, etc.).

According to various embodiments herein, in general, a biological polymer, such as a nucleic-acid molecule from a sample of interest, is maintained on or proximate to a support element, such as a particle or bead. In various embodiments, the support element is fixed or immobilized; in a variety of embodiments the support element is, or can be, mobile (moving, or adapted for movement). The support element and associated nucleic-acid molecule are disposed such that at least two opposing forces, when active, can act upon the support element. At least one of the forces has a dependence on the number of monomers of the nucleic acid molecule. The support element tends to a normal state (e.g., a generally fixed or otherwise an initial location or velocity) within an environment and/or system about it, which includes the opposing forces and any charges associated with the support element or otherwise influentially proximate it (for example, charges of or relating to the nucleic-acid molecule). The nucleic-acid molecule is then changed in a fashion effective to change the balance of the forces acting on the support element, consequently effecting a change in a detectable property of the support element (e.g., the perturbation can move the support to a different location or can change the velocity of the support). Detection and measurement of the change in the affected property of the support element can indicate at least one characteristic or aspect relating to the sequence of the nucleic-acid molecule. In various embodiments, DNA is bound to the support element.

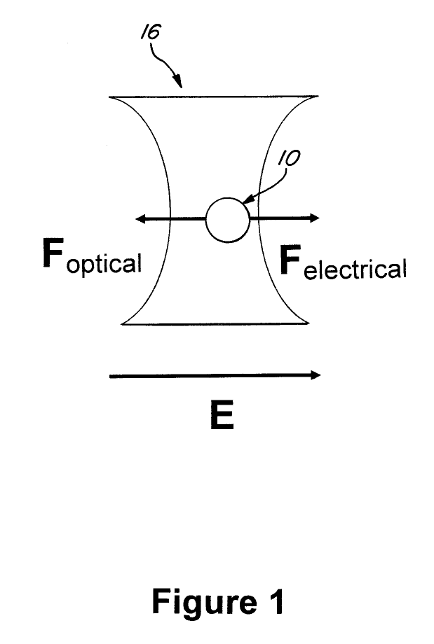

In various embodiments, systems, apparatus, devices, components, and the like, are contemplated by the teachings herein. Referring now to FIG. 1, various embodiments of such systems (etc.) can include, e.g., at least two opposing forces, including an electrical force, such as F_{electrical}, and one or more opposing forces, such as an optical force F_{optical} of an optical trap, denoted as 16, acting on a particle, as denoted at 10, can be used to generate a signal parameter whose value depends on the number of charges on the particle. The number of charges can be changed, for example, by synthesizing nucleic acid, cleaving nucleic acid, binding an oligonucleotide, displacing a bound oligonucleotide or ligating nucleic acids to a strand of nucleic acids (not shown) bound to the particle. Employing a detection and measurement system (not shown), re-measurement and analysis of the signal parameter which depends on the charge of the particle can be used to determine the sequence of the nucleic acid.

As mentioned, various embodiments herein contemplate a sequencing-by-synthesis (SBS) approach as means to change the size of a nucleic-acid molecule (e.g., an oligonucleotide) immobilized at one end to a solid support element, such as, e.g., a polystyrene bead or a gold particle. It is noted, at this point, that according to some SBS approaches, DNA polymerase or ligase enzymes can be used to extend a DNA strand, or a plurality of DNA strands (e.g., in parallel) or RNA polymerase can be used to extend RNA strands or reverse transcriptase can be used to extend DNA strands. Nucleotides or short oligonucleotides (e.g. DNA, RNA, PNA etc.) are provided either one at a time, or modified with identifying tags, so that the base type of the incorporated nucleotide or oligonucleotide can be determined as extension proceeds. SBS approaches, in general, may be categorized as either single-molecule-based (involving the sequencing of a single molecule) or ensemble based (involving the sequencing of multiple identical copies of a DNA molecule, typically amplified together, as on isolated surfaces or beads). They may be real-time (that is, with a free-running DNA polymerase given all nucleotides required) or synchronous-controlled (that is, using a priori temporal information to facilitate the identification process in a ‘stop-and-go’ iterative fashion). This can be achieved, for example, by using nucleotide substrates that are reversibly blocked or by simply adding only a single kind of nucleotide (e.g., dATP) at a time. (Carl W Fuller et al., “The challenges of sequencing by synthesis,” Nature Biotechnology 27, no. 11 (11, 2009): 1013-1023.)

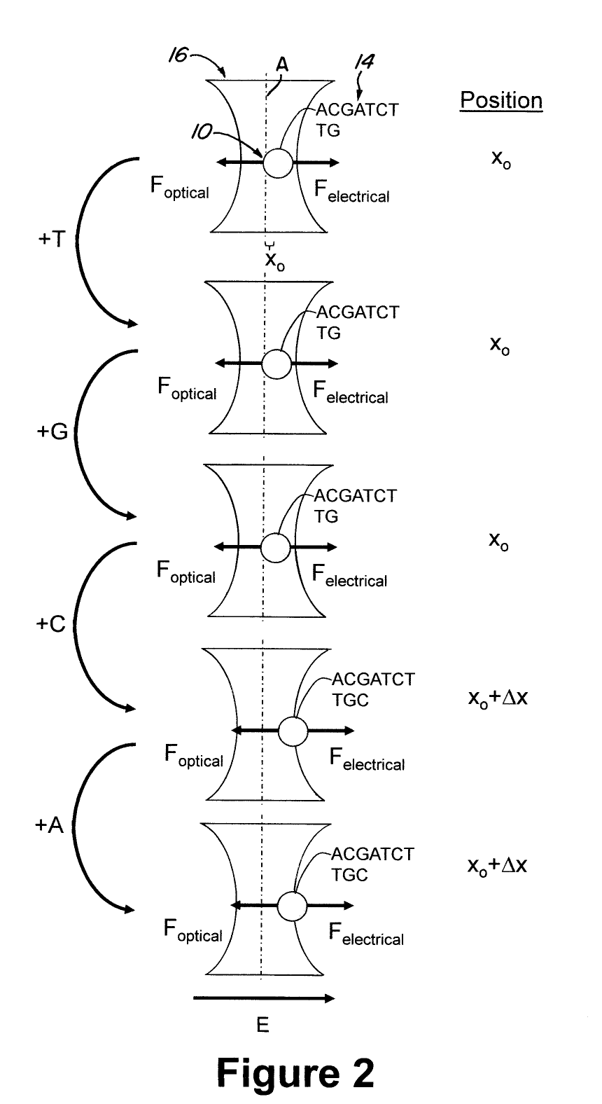

FIG. 2 shows an optical trap and electrical field for example, a suitable DNA polymerase (not shown) can be used to synthesize nucleic acids in the presence of one nucleotide at a time in such a way that changes in the resulting position of a support element, such as a particle or bead, can be used to determine the sequence of the nucleotide. For example, a support element, such as a particle 10, associated with a nucleic-acid molecule of interest (e.g., a strand of DNA, RNA, or the like), such as DNA strand 14 (which can be primed at its 3′ end), can be disposed in an electric field, as indicated by arrow E (which extends in the direction of arrow E), in an optical trap, 16 (such as described in connection with FIG. 1), wherein light can apply forces to the particle. At his point, it is noted that an optical trap can be created, for example, by tightly focusing a laser beam or by opposing fiber optics. According to various embodiments herein, forces can be applied, for example, so that the position of the particle tends to a location in an aqueous medium due to at least one restoring force.

With continued reference to FIG. 2, and, particularly, with attention to the sequence of processing steps shown, from the top of the figure downward to the bottom, it can be seen that upon incubating the particle-bound DNA 14, separately and sequentially, with each of four different mixtures (under conditions suitable for polymerase-mediated extension), wherein each mixture includes: (i) reagents appropriate for a polymerase-mediated extension reaction and (ii) one (and only one) of the four natural nucleoside triphosphates (dNTPs) for DNA [note, in the FIG. 2, it can be seen that four mixtures, each including only one of the four natural nucleotides, T, G, C, and A, respectively, are provided, separately and sequentially, to the particle-bound DNA 14]; upon changing the number of nucleotides (as can be seen, in FIG. 2, to take place upon incubating the particle-bound DNA 14 with a mixture including appropriate extension reagents, but only C insofar as deoxynucleotides are concerned), a detection and measurement system, including, e.g., a quadrature photodetector, (not shown) can be employed to detect and measure a consequent physical change in an affected (i.e., by the extension event) property of the particle. Notably, in FIG. 2, from the top of the figure, particle 10 can be seen to remain located at a position xₒ, directly adjacent a line, denoted as A, extending along a vertical axis of optical trap 16, as the deoxynucleotides, first, T, then second, G, are incubated, separately and sequentially, with the particle in distinct mixtures. When, third, the deoxynucleotide, C, is incubated with particle 10 in another distinct reaction mixture, the deoxynucleotide C (being the complement of G; as can be seen, the next nucleotide for determination in the figure) is incorporated in an extension reaction, at which point the balance of forces changes, causing particle 10 to move rightwards (when looking at the figure), until once again the various forces balance and it locates at a new position, xₒ+ΔX (as can be seen towards the bottom of FIG. 2). Upon incubation of particle 10 with a forth distinct reaction mixture, including appropriate extension reagents, but only the deoxynucleotide, A, insofar as deoxynucleotides are concerned, particle 10 remains at position xₒ+ΔX, as A is not the complement of the next base to be determined, which is also A in FIG. 2, so no extension event takes place.

It should be noted that, according to various embodiments, care is taken to know which nucleotide is present in each of the four reaction mixtures, as each of the four is incubated with the particle. In this way, upon observing movement of the bead (or other appropriate parameter), resulting from an incorporation event, the identity of a previously unknown base, awaiting determination, can be readily known. Once a base of nucleotide 14 has been determined, the process can be repeated in order to determine the next base, and so on. Thus, as depicted in FIG. 2, at least two opposing forces, such as F_{electrical}, and one or more opposing forces, such as optical force F_{optical}, acting on a particle, such as 10, which includes at least one nucleic-acid molecule, such as 14, bound to it, can be used to generate a signal parameter which can be employed to determine the incorporation by a polymerase of specific nucleic acids onto a primed polynucleotide bound to a particle.

In various embodiments of the present teachings, at least two forces are used, including a first force and a second force, with the second force opposing the first force. Forces oppose each other when the forces have a vector component in opposite directions. At least one of the forces, in a variety of embodiments, strongly depends on the number of monomer units in the biological polymer of interest. In some embodiments, one or more forces in addition to the second force also oppose the first force. In many embodiments, it can be advantageous to include an electrical force among the forces employed as a means of applying a force which depends on the length of a charged polymer of interest. In various embodiments, including certain embodiments described next, at least three forces are employed, including (i) an electrical force, and (ii) two additional forces, with one of the latter forces being a restoring force (e.g., a “spring-like” force).

In various embodiments, a strong dependence on the number of monomer units in the biological polymer of interest is determined to exist when a plot of the force versus charge (or length, or monomer number) would show a high slope; whereas, a force with a weak dependence which would show a low slope. Thus, in a variety of embodiments, “strong” and “weak” are considered relative to one another in a given system. And, as stated elsewhere herein, in various embodiments the one or more forces, other than the force that exhibits a dependence, show only very little to no such dependence. In some embodiments, where a strong force is indicated by a plot of the force versus charge (or length, or monomer number) showing a high slope, such high slope is relative to a force with a weak dependence which shows, for example, about ⅓ or less, about 1/10 or less, about 1/100 or less, the slope for the strong dependence.

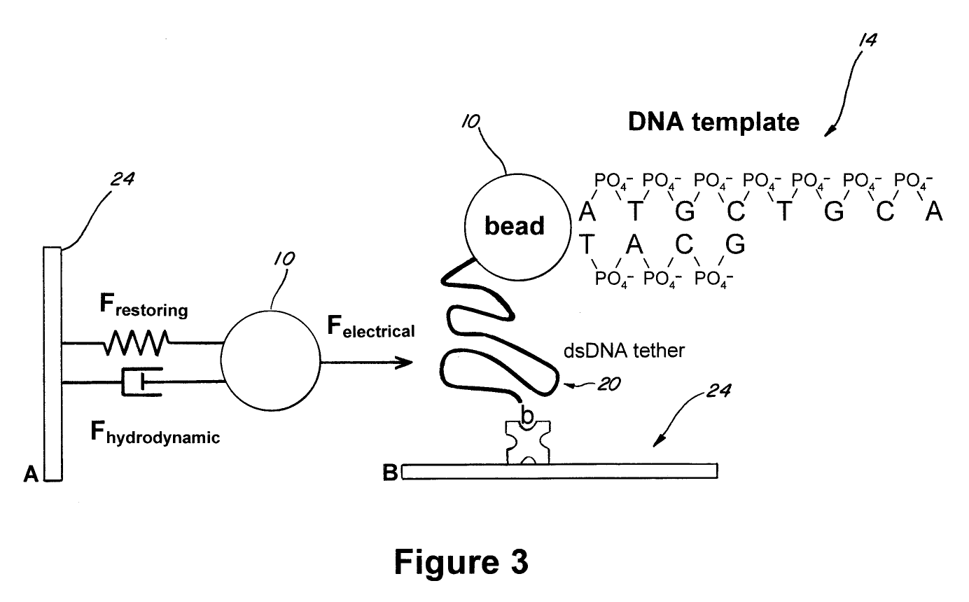

For example, FIG. 3 shows, in schematic form, a bead 10, and three forces which can act on the bead. FIG. 3(A), in particular, schematically depicts the three applied force vectors. As shown, the forces can include (1) an electrical force, F_{electrical}, characterized by being directly dependent on the total (net) electrical charge of DNA on the bead; and, additionally, (2) two opposing forces comprising (a) a hydrodynamic force (F_{hydrodynamic}), and (b) a restoring force, F_{restoring}.

In various embodiments, the restoring force is “spring-like” in nature, and connects or links a bead or particle to an appropriate surface. In FIG. 3, for example, tether 20 is attached to a suitable surface, 24, by any suitable means. In some embodiments of the present teachings, a polymeric molecule, such as a biological polymer, is employed as a tether providing a restoring force. With reference to FIG. 3(B), various embodiments contemplate employing an oligonucleotide as a tether providing a restoring force; such as double-stranded DNA (dsDNA) 20. Various embodiments contemplate other biological polymers, as well. For example, a tether can comprise a protein providing a restoring force. Various embodiments contemplate that a tether can be comprised of one or more non-biological polymers. For example, a tether can comprise polyethylene oxide.

FIG. 3(B) shows additional aspects of the bead 10 shown in FIG. 3(A). For example, one or more DNA templates, such as DNA template 14, can be attached at, or near, one of its ends to bead 10. The bead, in turn, can be attached to a surface, such as 24, via a suitable tether 20. In various embodiments, no more than a single DNA template is attached to a particle or bead. In some embodiments, one or more particles or beads can be coated, covered, spotted, etc., with clonal copies of selected DNA template, and attached to a surface via a means for tethering. In various embodiments, wherein a plurality of beads is used, the beads can comprise an array (e.g., a regular array), as desired.

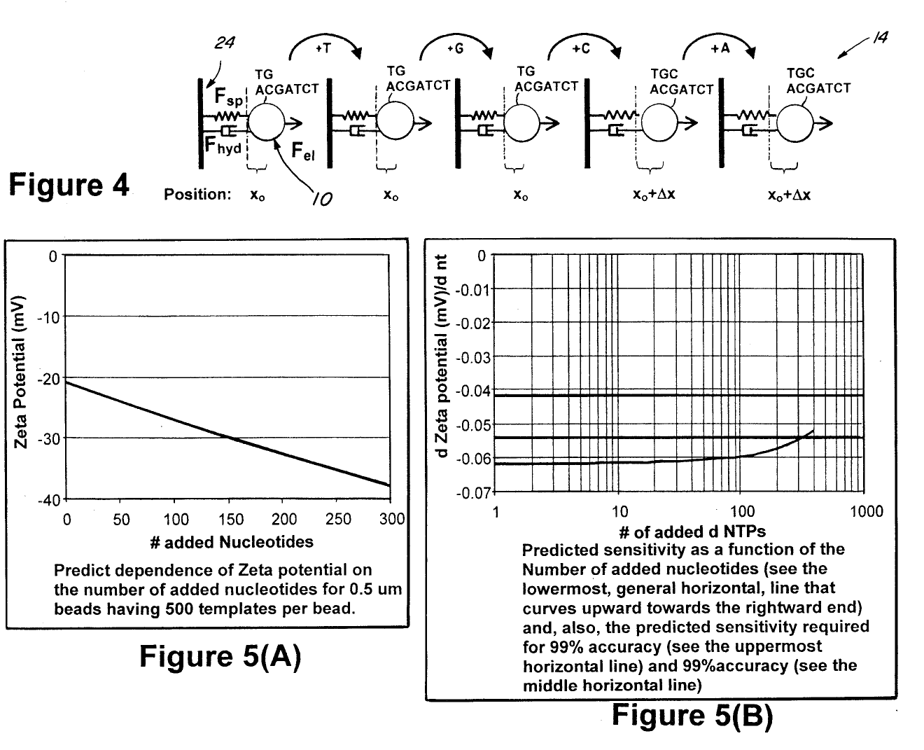

In practice, an analysis (e.g., sequencing) can be carried out using nucleic acid-bearing beads, in the system of FIG. 3. A process, e.g., such as described above and depicted in connection with FIG. 2, can be carried out employing the system of FIG. 3. In this regard, reference is now made to FIG. 4, which schematically depicts such as process using the system of FIG. 3. In FIG. 4, an electrical force, Fₑₗ, and two opposing forces, a hydrodynamic force, F_{hyd}, and a spring-like restoring force, Fₛₚ, act on bead 10. One round of sequencing-by-synthesis (SBS) is depicted in FIG. 4; that is, incubation of DNA template, 14, bound to particle, 10, under conditions suitable for polymerase-mediated extension, separately and sequentially with each of four different mixtures, wherein each mixture includes reagents appropriate for a polymerase-mediated extension reaction; and, one (and only one) of the four natural nucleoside triphosphates (dNTPs) for DNA. It will be appreciated that with each sequential addition of a nucleotide a change takes place in the balance of forces, leading to a detectable, measurable change in a bead parameter, e.g., position or velocity.

In various embodiments including at least two forces additional to a first (electrical) force, such as the hydrodynamic and restoring forces shown in FIG. 3, do not depend strongly on the number of nucleotides of the nucleic acid on the bead surface. Rather, in some embodiments, they depend only weakly on the number of nucleotides of the nucleic acid on the bead surface, and in various embodiments the additional forces do not exhibit any dependence on the number of nucleotides of the nucleic acid on the bead surface.

In various embodiments, tethered polymers can be used as “springs” to hold beads proximate a surface. In some embodiments, bead arrays are formed by attaching a plurality of beads, each of which includes a biotinylated polyethylene glycol (PEG) linker, to a surface which is patterned with streptavidin. The streptavidin can be printed by any suitable means, such as by soft lithography printing methods. In some embodiments, the beads of an array can be biased to one side with a DC component of an electric field used during detection, thereby stretching the respective tethers to yield spring constants of ˜0.1 pN/nm Bead motion can be detected in a variety of ways. In various embodiments, for example, an imaging array detector (e.g. CCD or CMOS cameras) can be employed, or a quadrature photodetector.

The restoring, spring-like force can be provided by any suitable means; for example, in a variety of embodiments, a restoring-force means comprises a tethering means or structure. Tethering means, as contemplated in various embodiments, can be selected so as to have a “spring-like” nature or characteristic. In various embodiments, tethering means can be selected or adapted to be functional for linking a particle, or bead, to a separate nearby structure. Tethering means can be selected, according to various embodiments, to span a region separating a DNA-bearing particle and a solid support, such as a nearby surface feature, such as 24 in FIG. 3. In a variety of embodiments, a means for tethering connects, or links, a nucleic acid-(including, e.g, DNA, RNA or PNA or modified nucleic acids such as locked nucleic acids, or the like) bearing particle with a solid support, such as a surface of a device, e.g., a surface or feature inside a flow channel, reaction chamber, and the like (e.g., in various embodiments, in a microfluidic device). In various embodiments, tethering means can be selected or adapted to be at least somewhat resiliently flexible. In various embodiments, a means for tethering is selected or adapted to be substantially stable under conditions appropriate for, and/or upon occurrence of an event effective for changing the size or length of a nucleic-acid or other molecule, such as a DNA template, associated with a particle or bead (e.g., adding or removing nucleic acids). In some embodiments a means for tethering is stable under conditions appropriate for SBS. In various embodiments, tethering means are selected for characteristics such as low cost, and if desired, suitability for fabrication of large bead arrays for high-throughput sequencing experiments. In some embodiments, with regard to the latter, polymer tethers can be employed.

At least one of the forces depends on the number of monomer units in the biological polymer of interest. It is contemplated herein that the choice of such forces, their directions of application, and the magnitude of their strength can vary. The forces can be, but are not limited to, body and surface forces acting on the nucleic-acid molecules and/or on the support. In various embodiments, an electrical force, which depends on the number of nucleic acid charges associated with the beads, is opposed by a hydrodynamic force on a bead and a force induced by stretching a polymer tethering the bead to a surface. Sequencing can be accomplished, for example, by optically monitoring changes in the motion of the bead as the balance of the forces changes with sequential incorporation of nucleotides.

It is contemplated herein that the choice of a force or forces opposing the one or more force which depends on the number of monomers of the polymer of interest and their directions of application can vary. According to various embodiments, suitable forces can include, for example, magnetic forces (e.g. magnetic tweezers), electrical forces (e.g. electrophoretic, dielectrophoretic), optical forces, hydrodynamic forces, entropic, mechanical, gravitational or centrifugal forces. In many embodiments, the configuration of opposing forces is selected to enhance detection sensitivity, accuracy, precision, and/or speed of measurement.

In some embodiments, forces are applied in a way to generate a spatially separated array of particles; which, among other things, can facilitate multiplexing of analysis. For example, optical, magnetic, and dielectrophoretic forces can be useful for creating arrays of particle traps, as will be understood to those skilled in the art. The arrays can comprise, for example, more than 1, more than 100, more than 10,000, more than 1,000,000, and/or more than 10,000,000 particles. In other embodiments, for example, an array of polymers is spotted onto a substrate. Such polymers can, for example, bind to the substrate and the particles and generate an entropic force to oppose an electrical force.

According to various embodiments, the forces can be applied in a variety of ways. For example, in some embodiments, a time-varying force (e.g. an AC voltage) can be applied to enable the use of Fourier techniques to facilitate precision in measuring the particle parameter. In other embodiments, for example, a force can be turned on and off quickly and the response of the particle parameter to this impulse measured. In further embodiments, for example, the forces can be applied continuously or in a steady-state manner.

With regard to means for effecting a change to one or more nucleic-acid molecules associated with one or more particles of the present teachings, various means are contemplated herein. For example, means for changing the number of monomers of nucleic-acid molecules can include: (i) extension of one or more strands, such as by polymerase-mediated synthesis; (ii) cleavage of one or more strands, as by any suitable enzyme possessing an appropriate nuclease activity; (iii) binding one or more oligonucleotides to one or more nucleic-acid molecules bound to the particle; (iv) displacing one or more oligonucleotides bound to one or more nucleic-acid molecules bound to the particle; (v) synthesis of a nucleic acid strand as by RNA polymerization, DNA polymerization or reverse transcription; or (vi) ligating one or more nucleic-acid molecules to one or more nucleic-acid molecules bound to the particle.

Other means for effecting a change to one or more nucleic-acid molecules associated with one or more particles can be employed, as well. For example, a polymerase, primer and template bound to the particle can be used. In some embodiments, similar to the sequencing by synthesis approach used in a commercially available pyrosequencing system (namely, the GENOME SEQUENCER FLX SYSTEM (454 Life Sciences, a Roche Company; Branford, Conn.)), solutions containing only one of the four nucleotides are added to the particles sequentially. Measurement of the particle parameter after each reaction step is used to determine whether one or more of a given nucleotide is incorporated. In another example, a polynucleotide bound to the particles is labeled with charges such that each different nucleotide has a different net charge (See, e.g., U.S. Pat. No. 6,780,982 B2, incorporated herein by reference). In the presence of a nuclease, nucleotides are sequentially released. By continuously monitoring the particle parameter, the identity of the released nucleotide can be determined.

Various embodiments contemplate chemical cleavage as means for effecting a change to one or more nucleic-acid molecules associated with one or more particles.

It should be appreciated, at this point, that opposing-force sequence analysis, as taught herein, includes a variety of embodiments that do not require or employ the use of labels on bases (i.e., label-free sequence determination using opposing forces), as well as embodiments that employ labels on bases, such as the charge labels just described (i.e., charge-label-based sequence determination using opposing forces).

Turning now to supports, a support associated with at least one nucleic-acid molecule, according to various embodiments, can be disposed such that a plurality of opposing forces can act upon it. The support element can be comprised of any of a variety of suitable materials and, as well, be configured in any suitable shape or form. Informed by the teachings herein, suitable support elements can be selected by those skilled in the art.

It is noted that, in connection with supports, no particular distinction is intended between the terms “particle” or “bead,” or the like; unless otherwise clear. Such terms, herein, may sometimes be used interchangeably. It is understood that the term “bead” can connote, in many instances, a structure that is generally spherical in overall dimension. Beads, so configured, are contemplated by a variety of embodiments herein. As well, the present teachings contemplate a variety of embodiments employing beads (and the like) of various other shapes. For example, without limitation, beads for use herein include entities or constructs that are semi-spherical, oval, oblong, globular, granular, flake-like, pellet-like, etc. Beads, particles and the like, according to various embodiments, can comprise, e.g., organic and/or inorganic materials. Beads, particles, and the like, contemplated for use herein, can comprise entities or constructs which are solid (in whole or in part), substantially solid, and/or semi-solid. In some instances, beads, particles, and the like, may be gelatinous or fluid, at least in part. Various embodiments of the present teachings contemplate beads, particles, and the like, such as those comprised of glass, quartz, polymers, or a combination thereof. A variety of embodiments herein contemplate beads, particles, and the like, comprised of metallic materials (such as gold particles) and/or aromatic polymers (such as polystyrene beads). In some embodiments, beads, particles, and the like, comprise one or more of the following (instead of, or in addition to, the materials just mentioned): liposomes, polymers, nanocrystals such as quantum dots, and oil droplets. Quantum dots, according to certain embodiments, can be described, e.g., in U.S. Pat. Nos. 5,990,479 and 6,207,392 B1, and in “Quantum-dot-tagged microbeads for multiplexed optical coding of biomolecules,” Han et al., Nature Biotechnology, 19:631-635 (2001), incorporated herein by reference.

A variety of embodiments herein contemplate use of a plurality of supports (simultaneously and/or sequentially). It will be appreciated by those skilled in the art that beads, particles, or the like, of a population, may or may not be uniform or identical in all respects. For example, in a given population intended to comprise smooth, spherical beads, there may be differences (of varying degree) in one or more aspects of the beads, e.g., diameter, surface feature(s) (e.g., smoothness), etc. In these regards, it is noted that herein, various embodiments contemplate strict tolerances for beads, or the like, of a population, wherein the distribution among and between individual members of the population is narrow. Some embodiments, contemplate a narrow distribution among plural populations themselves (e.g., embodiments wherein batches of nucleic-acid bearing beads are analyzed in parallel and/or series). In a various additional embodiments, wherein tolerances are not as strict, the distribution among and between individual support elements can be wider, though still relatively narrow. In other embodiments, the distribution may be wide.

As indicated, those skilled in the art can select suitable supports, being guided by various characteristics, purposes, functions, and such, relating to support elements, as taught herein. For example, criteria in selecting a suitable support can include, among other things, the size and/or binding capacity for nucleic-acid molecules associated with the support; the nature or purpose of the support in such association (e.g., a means for immobilizing, or otherwise maintaining within a desired location or orientation, one or more nucleic-acid molecules); the charge(s), if any, of the support itself (particularly when exposed to conditions effective to change a property or characteristic of one or more nucleic-acid molecules associated, or to be associated, with it).

It is noted that the size of a support, as contemplated herein, can vary. For example, a support, such as a bead, particle, and the like, can comprise a diameter (or mean diameter for one or more populations of beads), or other greatest dimension with regard to non-spherical support elements, within a range of: from about 10 nm to about 100 nm, from about 100 nm to about 1000 nm, from about 1 μm to about 10 μm, up to least about 10 μm, or greater. It should be noted that the charges of a support, itself, can vary; for example (in various embodiments), no greater than about 100, no greater than about 10,000, no greater than about 1,000,000. In some embodiments, the charges can be greater than 1,000,000. Some embodiments contemplate the use of gold particles, beads, or the like. The gold beads can, for example, be characterized by low surface charge, a single polymer tether, and, optionally, one or more capture primer oligos. In various embodiments, a plurality of gold beads include an average of 1 primed template, 1 tether, and, and less than 1,000 charges per bead. Discussed further elsewhere herein, in some embodiments employing gold beads, sequencing by synthesis is employed with DNA polymerase to obtain sequence from a gold bead with a single DNA template (producing, for example, greater than 10 nt, greater than 25 nt. and/or greater than 50 nt of sequence per bead with an accuracy at least about 90%, or greater).

Additional factors, which can be useful in the selection of supports, can include, for example, properties important to the interaction of the support with the selected forces and/or with the detection approach employed, such as density, dielectric constant, scattering cross-section, fluorescence, phosphorescence, polarizability, magnetic susceptibility, and/or electrical conductance. Furthermore, other considerations such as ease in surface modification, stiffness, surface energy, and barcoding capability (for example, to encode the identity of the source of the nucleic-acid molecules on the support) can also be relevant.

Some embodiments contemplate methods and systems employing no more than a single particle. That is, no more than one particle, associated with one or more nucleic-acid molecules, is acted upon by opposing fields for analysis (e.g., determining sequence information, in accordance with techniques taught herein). In some embodiments employing no more than a single particle, e.g., embodiments directed towards or otherwise useful for single-molecule sequencing (SMS), a particle (e.g., a bead) can be associated with no more than a single nucleic-acid molecule. In other embodiments employing no more than a single particle, on the other hand, plural nucleic-acid molecules can be associated with the single particle (such as a bead). Regarding the latter, some or all of the plural nucleic-acid molecules associated with the single particle are clones (that is, replicates of a nucleic-acid molecule of interest).

Various additional embodiments contemplate the use of plural particles. For example, at least about 10, at least about 50, at least about 100, at least about 1,000, at least about 10,000, at least about 100,000, at least about 1,000,000, and/or at least about 10,000,000 particles. For such embodiments utilizing plural particles, the particles can be disposed in any suitable arrangement, whereby opposing forces can act upon them and changes in a property of the beads can be detected.

In a variety of embodiments, a plurality of particles is configured so as to define an array. A variety of embodiments herein include particle arrays. The quantity of particles included in an array can vary. In various embodiments, for example, an array of particles includes at least 10, at least 50, at least 100, at least 1,000, at least 10,000, at least 100,000, at least 1,000,000, and/or at least 10,000,000 particles. The configuration of an array can vary. In various embodiments, arrays can be, for example, substantially planar. Various embodiments contemplate generally two-dimensional arrays. In some embodiments a plurality of beads are disposed in a linear array. In various embodiments, for example, a two dimensional array of beads is used such that the beads are in a repeating pattern with a pitch of between 0.1 and 20 microns. In other embodiments, for example, the beads can be in an array where the beads are in a non-repeating pattern.

With regard to encoding, various embodiments contemplate a variety of encoding means. Suitable encoding means can be selected by those skilled in the art. Encoding means can include, for example and without limitation, encoded or labeled polymeric, ceramic, semiconductor, and metallic particles or beads, barcodes, barcoding schemes, encodable tags, encodable labels, molecular encoding, oligo- and polynucleotide-based encoding, etc. Codeable tags, or other encoding means, can be selected to be suitably “detectably different.” In other words, they can be selected so as to be distinguishable from one another by at least one detection method. In various embodiments, detection of a given codeable tag, for example, can indicate the presence of a respective moiety to which the codeable tag is specific; while the absence of a given codeable tag can indicate the absence of the moiety to which the codeable tag is specific.

Information encoded, for example, can be specific to a particular moiety (or, in some instances, more than one moiety). In various embodiments, the identity of the source of a nucleic-acid molecule on a support element can be encoded. In some embodiments, this can be done, for example, for a plurality of different molecules of interest, such as from unique sources or samples.

In various embodiments, encoding means are employed with a plurality of bio-polymer (e.g., nucleic-acid molecule) carrying beads. In some embodiments, such plurality of beads is arranged so as to define an array (e.g., a planar array). According to a variety of embodiments, highly multiplexed analyses can carried out, substantially simultaneously (that is, in parallel). As well, detection of results can be carried out in parallel (e.g., employing an imaging apparatus). In various embodiments, micrometer- and nanometer-dimensioned encoded particles, beads, or the like, capable of, or adapted for, carrying biological molecules can be miniaturized and employed for multiplexing in an array-based format. In some embodiments, employing uniquely encoded particles tagged with specific recognition probes, a small amount of sample can be analyzed simultaneously for a plurality of targets.

Further regarding encoding, analyte-carrying support elements (e.g., particles carrying one or more nucleic-acid molecules of interest), according to a variety of embodiments, can be coded via position or placement. According to various embodiments, a plurality of nucleic-acid carrying particles is arranged in an addressable array (that is, an array having a known carrier particle associated with a known location (address) in the array.

Further regarding supports, as well as polymeric analytes associated with supports, in some embodiments that employ plural particles, such as those directed towards or otherwise useful for single-molecule sequencing (SMS), at least some of the plural particles are associated with no more than a single nucleic-acid molecule. In other embodiments, plural nucleic-acid molecules can be associated with at least some of the particles. Regarding the latter, in a variety of embodiments, some or all of the plural nucleic-acid molecules can be clones (that is, replicates of a nucleic-acid molecule of interest).

Various embodiments of the present teachings contemplate the generation of a plurality of replicates, or clones, of an individual nucleic-acid molecule, such as a strand of DNA. In various embodiments, for example, an in vitro cloning technique can be employed. In this regard, emulsion PCR (e-PCR) and/or bridge PCR can be utilized as a means, among others, for generating plural copies of a given or selected nucleic-acid molecule (e.g., a target DNA sequence).

With regard to some embodiments that contemplate generation of a plurality of replicates or clones of an individual nucleic-acid molecule, it is contemplated herein that the number of copies of the target nucleic-acid molecule can vary, as appropriate and/or desired. For example, some embodiments provide for the generation of a desired or otherwise selected number of copies. Similarly, some embodiments provide for the generation of a number of copies falling within a desired, or otherwise selected, range. For example, while some embodiments contemplate use of no more than a single (i.e., one, and only one) copy of a target sequence; other various embodiments contemplate use of a plurality of copies of a target sequence, e.g., up to about 1,000 copies, up to about 10,000 copies, up to about 1,000,000 copies, or more. A set, or sets, of one or more clonal nucleic-acid molecules can be associated with one or more particles, beads, or the like, as appropriate and/or desired.

According to various embodiments, any suitable means can be employed to associate one or more selected nucleic-acid molecules, or templates, with one or more particles for analysis in accordance with the present teachings (e.g., sequence determination). Nucleic-acid molecules can associate with particles in any suitable manner. For example, in various embodiments, such association can be by means of physical adsorption, covalent bonds or non-covalent binding. In some embodiments, nucleic-acid molecules can be synthesized on beads (e.g., chemical synthesis). If desired, the particles can be enriched for those particles containing desired nucleic acids.

With further regard to means for associating one or more nucleic-acid molecules with one or more particles, according to various embodiments, a target nucleic-acid molecule can be carried, attached, bound, fixed, linked, or otherwise maintained proximate to a selected particle by means, for example, of one or more sequence-specific capture oligos (note, the term “oligo” is sometimes used as an abbreviation for “oligonucleotide”). In various embodiments, one or more nucleic-acid molecules can be sheared, size fractionated, ligated with adaptors and captured by oligos on particles which hybridize with the adaptors or non-covalently or covalently bound to the particles. In some embodiments, one or more nucleic-acid molecules are synthesized onto one or more beads, e.g., chemically synthesized. Some embodiments contemplate, for example, solution-based, light-directed parallel in situ oligonucleotide synthesis.

In various embodiments, association is effective to maintain a selected single nucleic-acid molecule (e.g., in some embodiments, one, and no more than one), or plurality of nucleic-acid molecules (e.g., in some embodiments, a set of clones) in fixed relation proximate or on one or more particles, as desired. In various embodiments, the association is maintained for at least a period of time permitting analysis in accordance with the present teachings.

Some embodiments herein contemplate a direct association between a nucleic-acid molecule and a particle. Some embodiments herein contemplate an indirect association of a nucleic-acid molecule with a particle. In various embodiments, one or more appropriate linker moieties are employed as a means for associating a nucleic-acid molecule and a particle. A variety of linkers can be utilized. For example, linkers, as contemplated by various embodiments here, can be stable or can be labile, as desired. In some embodiments, a cleavable linker associates a nucleic-acid molecule with a particle. For example, among other suitable types, photocleavable linkers (PC-linkers) can be employed. Some embodiments contemplate use of a photocleavable biotin (PC-biotin) reagent as a means for releasably associating a nucleic-acid molecule with a particle. For example, in some embodiments, a PC-biotin reagent can be conjugated to an appropriate material or species comprising a particle, such as a primary amine using standard and facile NHS chemistries. Subsequently, near-UV light can be used to break the link between the biotin and the particle, as desired.

In some embodiments, one or more suitable intervening or bridging structures and/or layers are employed between a nucleic-acid molecule and an associated particle. For example, in some circumstances, it can be desirable to have a nucleic-acid molecule held or maintained in spaced-apart relation with respect to an associated particle. The spaced-apart relation can be fixed, substantially fixed, or it can be variable, as desired. In various embodiments, one or more suitable modifications are made to a particle (e.g., derivatization of a surface structure) for purposes of facilitating attachment. In other embodiments, the bridging structure between a nucleic-acid molecule and an associated particle can contain information of the identification (i.e., a barcode) of the origin of the nucleic-acid molecule and/or of the particle. The barcode can be a unique nucleic acid sequence, for example.

It should be noted that, herein, a distinction is generally not made regarding the type of association, direct or indirect, nor whether a polymeric molecule is in physical contact with a support element, such as a particle, bead, or the like, or maintained in spaced-apart relation. An exception being only where made clear that a distinction is pertinent and so deliberately intended. Thus, in using terms (or forms thereof) such as “associated,” “bound,” “carried,” “attached,” “linked” or the like, with regard to one or more polymeric molecules and one or more support elements; only unless otherwise made clear, details as to the nature of the association, such as being direct, indirect, touching, spaced-apart, or the like, are not intended and such terms are not to be understood as being limiting in these regards.

At this point, aspects of detection will be described. In accordance with the present teachings, a detector or detection system can be employed to detect, for example, one or more signals, parameters, or signal parameters of one or more beads, particles, or the like. The signal, parameter or signal parameter can be measured by a variety of means including, for example, magnetic, electrical (e.g. Coulter counter), and/or optical detection. For example, according to various embodiments, an optical detection system can be employed to detect a signal relating to a property of a particle; and in some embodiments, a property for each of a plurality of particles (e.g., an array of particles). Detected properties can include, for example, position, velocity (linear or rotational), acceleration or net force on the particle(s).

Detectors or detection systems, according to various embodiments, can include or be operably linked to any suitable computer system, or other suitable logic device, e.g., via an analog to digital or digital to analog converter, for transmitting detected light data to the computer for performing various tasks, such as collection, analysis, manipulation, and storage of data.

Any suitable means for detecting signals (e.g., optical signals) can be utilized. For example, in various embodiments, the means for optical detection includes systems adapted for measuring transmitted, absorbed, scattered, polarized, phase-shifted and/or emitted light. In various embodiments, for example, microscopes for brightfield, phase contrast, DIC or fluorescence microscopy can be used. Various embodiments of the present teachings employ a means for optical detection configured to measure one or more particle parameters from an array of particles. Various embodiments of arrays, as contemplated herein, can include more than 1, more than about 100, more than about 10,000, and/or more than about 1,000,000 particles. Suitable detectors include, for example, light detectors, such as quadrant photodiodes, and in some embodiments, imaging systems, such as charged coupled devices (CCDs or other array detectors), and the like. In some embodiments, it is contemplated that non-optical means for detecting changes in particle property can be used to infer changes in the associated nucleic-acid molecules, such as by magnetic, electrical resistance, capacitance, impedance, and acoustics approaches.

According to various embodiments, a computer system, or other suitable logic device, can be programmed to convert detector signal information into assay result information, such as length or sequence of one or more nucleic-acid molecules of interest, or the like. In some embodiments, a computer system is programmed to interpret detector signal information and provide output corresponding to sequences of nucleotide bases. In other words, the computer system can be configured for base calling. Signal data and/or assay-result information can also be sent, as desired, to an output or storage device, such as a display device (monitor), a printer, and/or disk drive.

A variety of programming means can be utilized, in accordance with various embodiments of the present teachings. In a variety of embodiments, e.g., sets of instructions for selected process steps can be written, for example, using C programming language (e.g., in some embodiments, a program is written in C for calculating from a CMOS sensor images of one or more analyte-bearing beads, positional information). Other programming means can be employed, as well. In various embodiments, a control computer can integrate the operation of various assemblies, for example through a program written in an event driven language such as LABVIEW® or LABWINDOWS® (National Instruments Corp., Austin, Tex.). In particular, the LABVIEW software provides a high level graphical programming environment for controlling instruments. U.S. Pat. Nos. 4,901,221; 4,914,568; 5,291,587; 5,301,301; 5,301,336; and 5,481,741 (each expressly incorporated herein by reference) disclose various aspects of the LABVIEW graphical programming and development system. The graphical programming environment disclosed in these patents allows a user to define programs or routines by block diagrams, or “virtual instruments.” As this is done, machine language instructions are automatically constructed which characterize an execution procedure corresponding to the displayed procedure. Interface cards for communicating the computer with the motor controllers are also available commercially, e.g., from National Instruments Corp.

Some embodiments employ a software tool, previously described, that facilitates and automates feedback control of an optical trap for dynamic single-molecule tethered-bead studies (See, e.g., Steven J. Koch (University of New Mexico, Deptartment of Physics & Astronomy and Center for High Technology Materials), and Richard C. Yeh, (New York), “Versatile Control System for Automated Single-Molecule Optical Tweezers Investigations,” NY; Nature Precedings: dl:10101/npre.2010.4284.1: Posted 16 Mar. 2010; http://precedings.nature.com/documents/4284/version/1/files/npre20104284-1.pdf; incorporated herein by reference). The latter provides a versatile control system to automate single-molecule biophysics experiments. The method combines low-level controls into various functional, user-configurable modules, which can be scripted in a domain-specific instruction language. The ease with which the high-level parameters can be changed accelerates the development of a durable experiment for single-molecule samples. Once the experimental parameters are tuned, the control system can be used to repeatedly manipulate other single molecules in the same way, to accumulate the statistics to report results from single-molecule studies. (National Instruments LabVIEW 7.1 and D, and LabVIEW 6.1. versions, for example, are available from SourceForge, and are described on an OpenWetWare site: https://sourceforge.net/projects/tweezerscontrol/).

Among the various advantages of the present teachings, it should be noted that, in a variety of embodiments, sequencing can be accomplished without the use of extrinsic labels for detection (in other words, free of extrinsic labels, such as fluorophores). Thus, the cost of reagents for sequencing can be greatly reduced compared to known, conventional sequencing approaches. In contrast to known, conventional fluorescence-based detection systems, which generally require a narrow excitation source and high numerical aperture collection optics, various embodiments of the present teachings can employ simple, dark-field optics. An ordered bead array, according to various embodiments, can also facilitate efficient use of the pixels in the imaging detector. A system of the present teachings can cost, for example, substantially less than the prior or existing commercially available systems, thereby reducing the instrument amortization cost (e.g., by 2-10 fold) compared to other sequencing approaches and systems.

Various embodiments of the present teachings (e.g., certain embodiments providing for single-molecule opposed-force sequencing) can facilitate achievement of the “$1000 genome,” as they provide at least some, and in various embodiments all, of the attributes generally understood in the art as being desirable towards achieving ultra low-cost sequencing.

Those skilled in the relevant arts will appreciate, e.g., from the embodiments set out herein, and the examples that follow, that the present teachings provide for label-free, as well as label based in some embodiments (e.g., charge-label-based), nucleic-acid sequencing using, among other things, two or more opposing forces. Further, it will be appreciated that sequence information can be determined via observation and measurement of physical properties, such as motion or displacement of one or more analytes (e.g., one or more nucleic-acid molecules supported by particles or beads) as they are subjected to opposing forces, e.g., opposing electrical, hydrodynamic, and entropic forces.

As well, in view of the embodiments herein and the following examples, the skilled artisan will appreciate that, in various embodiments, the present teachings provide for highly parallel, accurate, long read-length, label-free sequencing by synthesis (SBS).

Of particular note, various embodiments of the present teachings (e.g., embodiments providing for opposed-forces, single-molecule sequencing (SMS)) can facilitate generation of sequence information at an unprecedented low cost, as such embodiments provide at least some, and in various embodiments all, of the attributes generally understood in the art as being desirable towards achieving ultra low-cost sequencing.

Among other things, sequence information can be determined by monitoring changes in a parameter of a support element, associated with an oligonucleotide of interest, as the balance of forces acting upon the support element changes with sequential incorporation of nucleotides. Various aspects of opposed-forces sequencing, as embodied in various methods and systems herein, can be employed, for example, in de novo sequencing of mammalian genomes.

Studies and first-principle calculations indicate that opposed-forces sequencing can significantly reduce the cost of de novo sequencing, as compared to prior, known, and commonly employed approaches. Various aspects of the present teachings can avoid, or otherwise overcome, certain key issues and challenges faced by other non-Sanger-based sequencing approaches. In this regard, various aspects of the present teachings provide for: (1) the use of a single DNA template per bead, which facilitates low-cost sample preparation; (2) label-free detection, which avoids various costly sequencing reagents used in various other sequencing systems; (3) relatively simple optics, comprised of readily commercially available components, thereby providing for a relatively low instrument cost; (4) highly parallel sequencing (as by way of bead arrays), which provides for high throughput and, consequently, a low capital equipment amortization cost per genome; and (5) longer read lengths as compared to other non-Sanger-based sequencing approaches, which substantially reduces otherwise highly resource-intensive bioinformatics costs for genome assembly. Low-cost sequencing systems and methods realizing some or all of the just-mentioned benefits (e.g., various embodiments of opposed-forces sequencing systems and methods, herein) will greatly benefit medical research and clinical care.

### EXAMPLES

The present teachings may be further understood in light of the following examples, which are illustrative and are not intended in any manner to limit the scope of the present teachings or claims directed thereto.

### Example 1

### Part A

This portion of the present illustrative example describes illustrative studies, calculations and modeling to assess detecting the incorporation of nucleotides onto bead-bound templates. A microscope system capable of microelectrophoresis measurements is used to measure the movement of beads in response to opposing electrical and hydrodynamic forces. The system is used to measure the changes in Zeta potential as a result of the extension of primed DNA templates.

Zeta Potential Measurement

Here, illustrative studies are described to measure a bead charge (which effects Zeta potential) by microelectrophoresis.

Streptavidin-coated polystyrene beads (1-3 μm radius, Bangs Laboratories) are loaded by pressure-driven flow into a planar quartz microfluidic chip. An electric field (˜10 V/cm) is applied to the wells of the chip. Field polarity is reversed every half second. The channels are pretreated with a dilute sieving gel to minimize electroosmotic flow. The beads are imaged on a microscope (40× objective) using bright-field illumination, and images are captured on an CCD camera at 30 Hz, e.g., for 5 seconds. ImageJ (Abramoff, M. D., Magelhaes, P. J. and S. J. Ram. “Image Processing with Image)”. Biophotonics International, 2004, 11:36-42) with the SpotTracker plug-in (Sage, D., F. R. Neumann, F. Hediger, S. M. Gasser and M. Unser. “Automatic Tracking of Individual Fluorescence Particles: Application to the Study of Chromosome Dynamics.” IEEE Transactions on Image Processing, 2005, 14:1372-1383) is used to convert images of individual beads to plots of bead velocity versus time. The Smoluchowski equation (Hunter, R. J. Zeta Potential in Colloid Science. Academic Press, New York, 1981) is used to convert the velocity and bead parameters to a Zeta potential:

Z=ηv/E∈ᵣ∈ₒ (1)

where Z is the Zeta potential, v is the bead velocity, E is the electric field, ∈ᵣ∈ₒ is the product of the dielectric constant and the permittivity of free space and η is the viscosity.

Observing Polymerization by Microelectrophoresis

Here, the system described above is used to observe the incorporation of nucleotides onto a bead-attached, oligo-primed template.

A biotin oligonucleotide primer (5′biotin-20 mer-3′) is hybridized to a template oligonucleotide (5′-43 mer-3′) and bound to the streptavidin-coated beads in PCR buffer at room temperature for 30 minutes. Beads are washed and resuspended in PCR buffer containing either: (1) 200 μM dNTPs, (2) polymerase (0.025 U/μL), or (3) 200 μM dNTPs and polymerase (0.025 U/μL). Beads are incubated at 37° C. for 20 minutes, washed and resuspended in buffer containing 10 mM Tris (pH=8) and 50 mM NaCl. The Zeta potential is expected to show a significant decrease (i.e. more negative) under polymerization conditions compared to the −polymerase and −dNTP controls.

Sequencing by Synthesis Using Microelectrophoresis

Here, an illustrative example for ascertaining the ability to perform sequencing by synthesis is set forth, using the model system described above.

Beads with bound templates (with a sequence of AAT immediately following the primed site) are mixed sequentially with dTTP, dCTP and all four dNTPs. For each bead aliquot, images of ˜20 beads are acquired and bead motion is converted to Zeta potential as described above. Results are predicted to show a statistically significant change in the Zeta potential, indicate addition of two nucleotides in the presence of dTTP. Subsequent addition of dCTP is predicted to cause no change in the Zeta potential. Addition of a mix of all 4 dNTPs is predicted to lead to an increase in Zeta potential ˜10× greater than that for dTTP addition, as expected from the addition of 21 nucleotides per template.

This example is illustrative to demonstrate the ability to use the bead surface charge or Zeta potential to monitor the incorporation of nucleotides on bead-bound templates. This example can be extended by increasing the precision of the measurement and sequentially adding all four dNTPs by anchoring beads to allow for single bead measurements. These improvements are predicted to yield a scalable, label-free detection method for inexpensively measuring high accuracy, long-read length sequences.

### Part B

This portion of the present illustrative example describes illustrative studies, calculations and modeling to assess detecting the incorporation of nucleotides onto bead-bound templates. A microscope system capable of optical trapping, electrophoresis and bead position measurements is used to measure the movement of beads in response to opposing electrical, optical and hydrodynamic forces. The system is used to measure the changes in Zeta potential as a result of the extension of primed DNA templates.

McLaughlin's Zeta potential model (See: Galneder, R., V. Kahl, A. Arbuzova, M. Rebecchi, J. O. Rädler and S. McLaughlin. “Microelectrophoresis of a bilayer-coated silica bead in an optical trap: application to enzymology.” Biophys J., 2001, 80:2298-309) can be used to predict the expected behavior of the proposed system when working with beads subject to AC electric fields, hydrodynamic drag and a restoring spring-like optical force. The model can be extended to describe the case of a discrete charged molecule on the bead surface to predict the behavior of a single molecule sequencing system.

Long Read Lengths are Predicted

The Guoy-Chapman theory can be used to model the dependence of the Zeta potential of a particle under specific experimental conditions:

Z=2k_{b}T arcsin h[σ/(8N∈ᵣ∈ₒk_{b}T)^{1/2}]/e (2)

where Z is the Zeta potential, e is the electron charge, k_{b}T is the product of the Boltzman constant and temperature, σ is the surface charge density, N is the concentration of ions and ∈ᵣ∈ₒ is the product of the dielectric constant and the permittivity of free space. The minimum detectable difference in the Zeta potential of a particle in a harmonic trap undergoing a sinusoidally-varying electrical force, limited by the Brownian motion of the particle, was derived by McLaughlin (Galneder, R., V. Kahl, A. Arbuzova, M. Rebecchi, J. O. Rädler and S. McLaughlin. “Microelectrophoresis of a bilayer-coated silica bead in an optical trap: application to enzymology.” Biophys J., 2001, 80:2298-309):

Zₘᵢₙ=[2k_{b}Tη/(3πrtₜₒₜ)]^{1/2}/(E∈ᵣ∈ₒ) (3)

where η is the viscosity and tₜₒₜ is the measurement time, E is the electric field and r is the particle radius. Together, these equations can be used to model the dependence of the performance of the proposed system as a function of certain experimental conditions.

FIG. 5(A) shows the result of using Equation (2) to model the dependence of the Zeta potential of a bead of radius 0.5 μm, with no initial charges, with 500 copies of a primed template of length 300 nt, in a buffer with a 30 mM concentration of monovalent ions, using a 1,000 V/cm field with a 1 second observation time. Parameters are chosen to satisfy the desired accuracy and to keep the force on the bead in the 10 pN range. Compared to the McLaughlin group's work, greater sensitivity is expected due to an increase in the magnitude of the electric field. Other sets of conditions (field strength, bead size, number of templates, etc.) yield similar performance. As expected, increasing the number of nucleotides leads to a more negative Zeta potential. FIG. 5B shows the use of Eq. (2) to model the dependence of the change in Zeta potential per added nucleotide as a function of the total number of added nucleotides. The predicted base calling rates are obtained by multiplying the minimum value from Eq. (3) by a factor of 2.3 to obtain a base calling accuracy of 99% and by a factor of 3 to obtain a base calling accuracy of 99.9%. As can be seen, under these conditions it is predicted that 300 nucleotides could be read at 99.9% accuracy with a measurement time of 1 sec. Similar to the GENOME SEQUENCER FLX SYSTEM (454 Life Sciences, a Roche Company; Branford, Conn.)), the present approach (of this example) involves discrimination of the number of nucleotides incorporated per nucleotide addition. In the GENOME SEQUENCER FLX SYSTEM, the error in the intensity measurement increases linearly with the number of repeats in a homopolymer leading to insertion/deletion errors in homopolymeric regions (Quince, C., A. Lanzén, T. P. Curtis, R. J. Davenport, N. Hall, I. M. Head, L. F. Read and W. T. Sloan. “Accurate determination of microbial diversity from 454 pyrosequencing data.” Nature Methods, 2009, 6:639-641). Because the error in the change in Zeta potential per nucleotide is predicted to be approximately constant over small number (e.g. ten to twenty) of added nucleotides, it is expected that the present method (of this example) will be able to accurately read through homopolymer repeats.

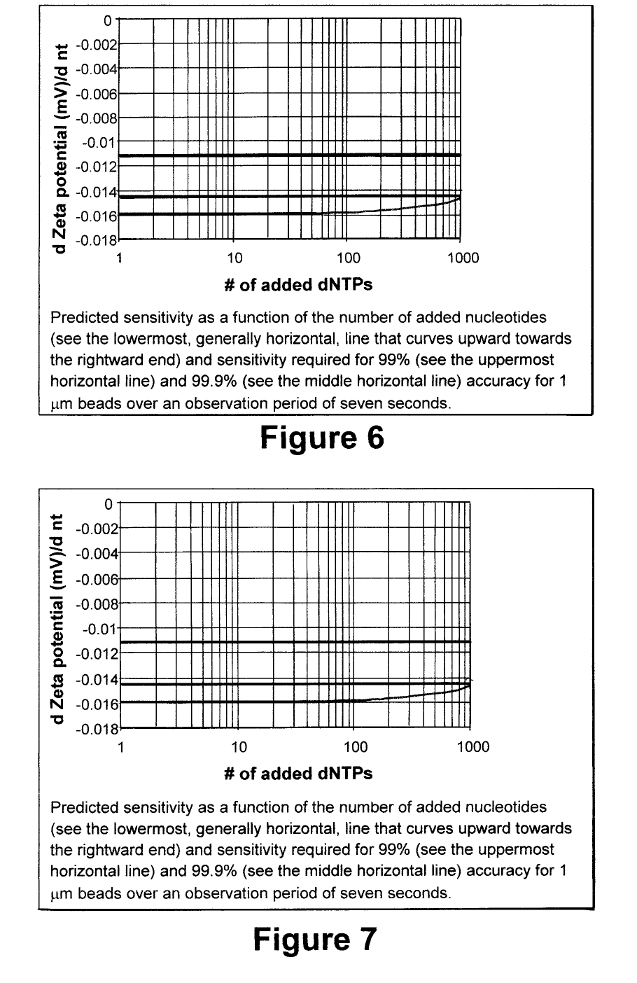

Model results indicate that read lengths longer than 1000 nt can be attained by using a larger bead and a longer measurement time. FIG. 6 shows the calculated sensitivity and read length using 1 μm radius beads and a 7-second measurement time. The larger bead adds more drag to the bead to keep the balance of forces in the 10 pN range and to reduce the noise from Brownian motion. Additional measurement time is required to offset the smaller change in Zeta potential per nucleotide. While the optical measurement may be capable of reads >1,000 bases, other limitations such as dephasing from asynchronous nucleotide addition may limit accuracy before this physical limit is reached (Mashayekhi, F. and M. Ronaghi. “Analysis of Read-Length Limiting Factors in Pyrosequencing Chemistry.” Anal. Biochem., 2007, 363: 275-287).

Towards Single Molecule Sequencing

To investigate the potential of sequencing with a single DNA template undergoing polymerization, the model is modified to directly describe the forces arising from the DNA molecule instead of relating the electrophoretic force on the bead to the Zeta potential. The force can be calculated as arising from shielded DNA charges in an electric field:

Fₑₗ=nq_{eff}E (4)

where n is the number of charges and q_{eff} is the effective charge due to ionic screening (See, Keyser, U. F., B. N. Koeleman, S. Van Dorp, D. Krapf, R. M. M. Smeets, S. G. Lemay, N. H. Dekker and C. Dekker. “Direct force measurements on DNA in a solid-state nanopore.” Nature Physics, 2006, 2:473-7). Under similar buffer conditions, the effective charge of a nucleotide was found to be 33% of e in Eq. (2). From the McLaughlin paper (See, Galneder, R., V. Kahl, A. Arbuzova, M. Rebecchi, J. O. Rädler and S. McLaughlin. “Microelectrophoresis of a bilayer-coated silica bead in an optical trap: application to enzymology.” Biophys J., 2001, 80:2298-309) the minimum detectable force is:

Fₘᵢₙ=[24k_{b}Tηπr/tₜₒₜ)]^{1/2} (5)

This model suggests that the additional force per nucleotide in a 2000V/cm field is 11 fN. If the DNA were attached to a neutral, 0.05-μm radius gold particle attached to the chip surface with a neutral dextran and observed for 6 seconds, the minimum force required for 99.9% accuracy is 10 fN. It is expected that polystyrene beads, if configured so small, would not scatter enough light required to be easily detectable. If, however, the bead is made from gold (as opposed to polystyrene), the amount of light scattered from a 0.25-μm bead is equivalent to that scattered from a 0.5-μm polystyrene bead (See, Yguerabide, J. and E. E. Yguerabide. “Light-scattering submicroscopic particles as highly fluorescent analogs and their use as tracer labels in clinical and biological applications.” Anal. Biochem., 1998, 262:137-56). This should allow detection with signal-to-noise equivalent to the polystyrene beads with multiple DNA templates. Taken together, the calculations suggest that it is a possibility to sequence using the present opposed-forces method (of this example) on a single copy of DNA per bead.

Attaining “$1000 Genome” Using Cost Estimates

Because no extrinsic labels need to be used for detection, the cost of reagents for sequencing is expected to be <$100 per genome sequenced. This assumes that samples are obtained as purified DNA and includes the cost of functionalized beads, attachment of templates to the beads, dNTPs, polymerase and the plastic disposable flow cell. Because the reagent cost is so low, the DNA sequencing cost is dominated by the amortization of the instrument cost. The amortization cost will decrease as the cost of the instrument decreases and as the DNA sequencing throughput increases.

Most of the cost of the instrument is expected to be dominated by the optical subsystem; for example: a high speed imaging area detector, illumination light, objective, and image processing computer. Compared to expensive fluorescence optics and the large number of pixels (>15) required to image one feature in the Pacific Biosciences system as described (See, Lundquist, P. M. et al. “Parallel confocal detection of single molecules in real time.” Optics Letters, 2008, 33:1026-1028), dark-field illumination and the lower number of pixels used per feature will lead to a low-cost instrument. Since images must be processed in real-time to extract the Zeta potential from the raw images, data must be processed at a rate of about 1 Gigabits/sec. Such rates are within what can be achieved using commercially available dedicated image processors such as FPGAs (See, e.g., Kehtarnavaz, N. and M. Gamadia. Real-Time Image and Video Processing: From Research to Reality. Morgan & Claypool, New York, 2006). Utilizing off-the-shelf components in the present system (of this example), the end-user price of the instrument is expected to be less than $100,000.

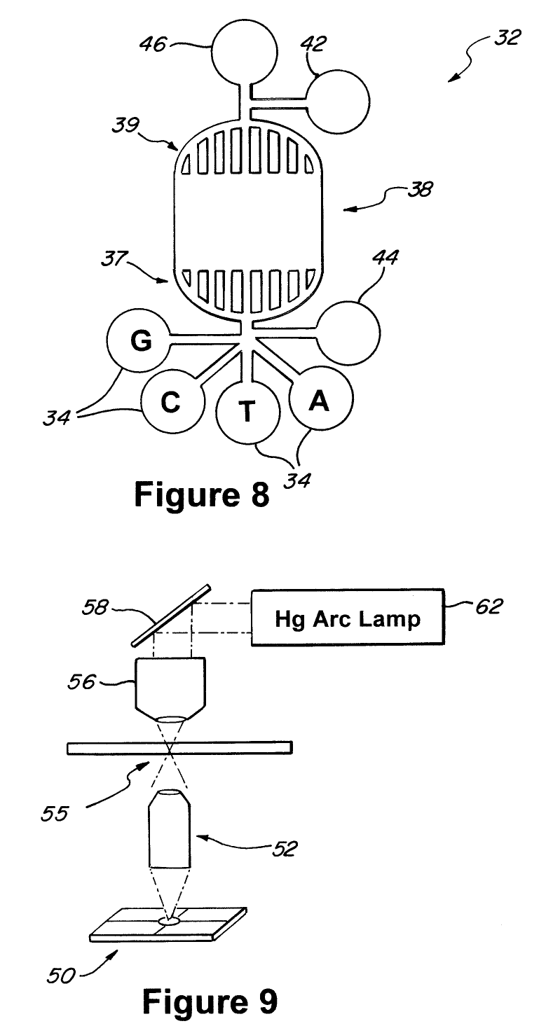

The throughput depends on the time required to add a new dNTP, the time required to image the bead motions, and the number of beads imaged in parallel. In the present system (of this example), a reduction in the dNTP exchange time is expected upon employing high-field electrophoresis to rapidly exchange the nucleotides in the detection area using a chip; such as chip 32 shown schematically in FIG. 8. Chip 32 includes a fluidic network with four separate reservoirs, denoted generally at 34, for introducing the 4 dNTP's (indicated, in FIG. 8, aptly by the letters G, C, T, and A) independently into a chamber, at 38, for beads (not shown). Chip 32 also includes anode and cathode buffer wells, 42 and 44, respectively; and a waste well, 46. Given the electrophoretic mobility of nucleotides (˜−3×10⁻⁴ cm²/Vs), a field of ˜1000 V/cm should allow exchange of the ˜0.5 cm long detection area (2 cm×0.5 cm) in <3 seconds. To ensure a uniform electric field throughout the detection area, the channels entering and exiting the detection area (indicated generally at 37 and 39, respectively) can be split into multiple, equal length channels. Although the high fields used for dNTP exchange and for oscillating the beads will require dissipation of ˜5 W/cm² of heat, active cooling from one side of a thin chip should allow the temperature (and thus viscosity) to be precisely maintained. As seen from the above section, the imaging time can range from 1 to 7 seconds depending on the desired read length. The combined time required for dNTP exchange and imaging can range from 16 to 40 seconds for one cycle of 4 dNTP addition.

High throughput sequencing entails measuring a large array of beads. Systems have been developed for forming arrays of tens of optical traps (See, e.g., Merendaa, F., J. Rohnera, P. Pascoalb, J. Fourniera, H. Vogelb and R. P. Salathéa. “Refractive multiple optical tweezers for parallel biochemical analysis in micro-fluidics.” Proceedings of SPIE, the International Society for Optical Engineering, 2007, 6483:64830 A.1-9), but there is no known clear path to attain a low cost array of hundreds of traps, thousands of traps, tens of thousands of traps, hundreds of thousands of traps, a million traps, or greater than a million traps. Rather than utilize such optical traps, the present opposed-forces sequencing system (of this example) contemplates use of a restoring force, which can comprise an “entropic force” exerted by stretching a polymer chain from its termini (See, e.g., Meiners, J. and S. R. Quake. “Femtonewton Force Spectroscopy of Single Extended DNA Molecules.” Phys. Rev. Lett., 2000, 84:5014-17). The entropic force exerted by the polymer is proportional to the end-to-end polymer distance (h) with a spring constant K:

K=3k_{b}T/<h²> (6)

where k_{b} is the Boltzman constant, T is temperature, and <h²> is the mean-square distance of the chain ends (See, Dutcher, J. R. and A. G. Marangoni. Soft Materials: Structure and Dynamics. Dekker-CRC Press, New York, 2004).

For example, the arrays can be generated by attaching beads to a patterned array of streptavidin with a biotinylated DNA linker (See, e.g., Zimmermann, R. M. and E. C. Cox. “DNA stretching on functionalized gold surfaces.” Nucleic Acids Res., 1994, 22:492-7). The patterned streptavidin array can be inexpensively printed onto the detection area with 30 nm resolution using soft lithography printing methods (See, e.g., Xia, Y. and G. M. Whitesides. “Soft Lithography.” Annu. Rev. Mater. Sci., 1998, 28:153-84). Using a small spot size relative to the bead diameter should insure that binding of only one bead per spot is favored. By biasing the beads to one side with a DC component of the field used during detection, the DNA linker can be stretched to the high force region for DNA. In this way, spring constants of ˜0.1 pN/nm (See, Smith, S. B., Cui, Y. and C. Bustamante. “Overstretching B-DNA: The Elastic Response of Individual Double-Stranded and Single-Stranded DNA Molecules.” Science, 1996, 271:795-9; and, Cluzel, P., A. Lebrun, C. Heller, R. Layery, J. L. Viovy, D. Chatenay and F. Caron. “DNA: an extensible molecule.” Science, 1996, 271:792-4), comparable to those obtained with optical traps (See, Simmons, R. M., J. T. Finer, S. Chu S and J. A. Spudich. “Quantitative measurements of force and displacement using an optical trap.” Biophys J., 1996, 70:1813-22), can be obtained. Under these conditions, the bead center is expected to be within ˜200 nm of the target location. The array of beads can thus be aligned with the detector array to center the beads at the desired detector location. The use of structured illumination can convert bead movement to intensity changes using a single pixel per bead (See, Gustafsson. M. G. “Nonlinear structured-illumination microscopy: wide-field fluorescence imaging with theoretically unlimited resolution.” Proc. Natl. Acad. Sci. USA, 2005, 102:13081-6). An even more robust approach can entail the detection of bead motion in a manner analogous to the quadrature photodetector approach commonly used to detect bead motion in optical traps (See, Svoboda K. and S. M. Block. “Biological applications of optical forces.” Annu. Rev. Biophys. Biomol. Struct., 1994, 23:247-85). Since bead motion is driven along the vector defined by the electric field, a bead positioned between two pixels can use just two pixels to detect bead motion. In a configuration where the spread of the bead image is less than the pixel size, no additional pixels are required to prevent inter-bead crosstalk. Using a 2-megapixel sensor would, thus, allow measurement of 1 million beads in parallel.

Additionally, the cost of genome assembly can be greatly reduced by using long read lengths (>300 nt). In order to attain sequence comparable to the draft mouse genome sequence (See, Chinwalla, A. T., et al. “Initial sequencing and comparative analysis of the mouse genome.” Nature, 2002, 420:520-562), it is contemplated with the present system (of this example) to use ˜7× coverage with a raw accuracy >99.9%. It is expected that the read lengths >300 nt will allow assembly with contiguity on par with that observed for the draft mouse genome. Combining these results, a $50K instrument, for example, amortized over three years operating at rate of 20 seconds per nucleotide for 1 million beads in parallel would be able to sequence 21 billion bases of DNA at an amortized instrument cost of ˜$200.

### Part C

Summary

Above, the ability to monitor incorporation of nucleotides into bead-bound templates by measurement of changes in the bead Zeta potential with sensitivity close to a single base is illustratively described. Model calculations indicate that, optimized, this method should enable accurate, long read length and label-free DNA sequencing. The lack of labels leads to extremely low reagent costs. The simple detection optics (no fluorescence) leads to a low-cost instrument. The high degree of parallelization will also reduce the instrument amortization cost per base. Long read lengths lead to low sequence assembly costs. The much lower per-bead copy number required, e.g., as compared to the GENOME SEQUENCER FLX SYSTEM (454 Life Sciences, a Roche Company; Branford, Conn.)), should allow for sample preparation options other than the laborious PCR procedures (such as emulsion PCR and bridge PCR) burdening the popular, present-day “next-generation” sequencing systems, making initial sample preparation easier and cheaper. Additionally, the present systems and methods (of this example) are contemplated for use in sequencing a single molecule (often referred to as “single-molecule sequencing,” “SMS,” and “third-generation sequencing”) wherein template amplification (e.g., PCR) is not performed in preparing a sample for sequencing, thereby even further reducing sample preparation costs.

### Example 2

### Introduction

This example provides an illustrative demonstration of systems and methods of sequence determination by use of opposing forces; and, is particularly illustrative with regard to (1) obtaining sequence from a single bead using an electrical force opposed by a hydrodynamic force and an entropic force from a tethered polymer chain; and, (2) a scalable detection approach that extending opposed-forces sequencing from a single bead to about one million beads, or more.

As will be seen, a number of items are addressed in connection with this illustrative example, including: (1) Generating neutral beads (<10,000 charges/bead) with attached template DNA; (2) Attaching beads to a surface with a single polymer tether; (3) Measuring Zeta potential using a quadrant photodiode system on a single bead with sensitivity <0.1 mV; (4) Sequencing on individual beads to 50 nt with 90% accuracy; (5) Confining of bead image to <9 pixels while acquiring images at >50 Hz; (6) Providing an image processing program for measuring Zeta potential with <1% precision.; (7) Sequencing to 75 nt at 90% accuracy using an imaging array sensor; and, (8) Applying optical parameters to system model to configuring a system and method for generating more than 2 GB of sequence information per day.

Part A: Opposed-Forces Sequencing Using a Polymer Tether

In this aspect of the present illustrative example, a polymer tether is made and used. The polymer tether comprises a single dsDNA tether linking a microsphere to a solid surface as the restoring force. The spring-like restoring force of DNA has been well characterized (see, e.g., Smith, S. B., Cui, Y. and C. Bustamante. “Overstretching B-DNA: The Elastic Response of Individual Double-Stranded and Single-Stranded DNA Molecules.” Science, 1996, 271:795-9; and, Cluzel, P., A. Lebrun, C. Heller, R. Layery, J. L. Viovy, D. Chatenay and F. Caron. “DNA: an extensible molecule.” Science, 1996, 271:792-4). Additionally, the use of DNA is conducive to customizing the length of the tether chain, and it provides a wide selection of linker chemistry. Next, an illustrative demonstration is provided of a single bead.

An Entropic Opposing Force Via Tethering Individual Microbeads onto a Glass Substrate with dsDNA

First, efforts are made to maintain the native charge of the microbeads close to neutral in order to maximize the sensitivity and dynamic range of the charge change measurements on the bead. Monodisperse polystyrene beads are used because they are nearly electrically neutral prior to adding functional groups on the surface, and are associated with a wide selection of commercially available linker chemistry. Second, efforts are made to tether the microbead (in this example) through no more than a single DNA chain so that the stiffness of the entropic spring is well-controlled. Finally, the tethering protocol is characterized by compatibility with a low-copy number clonal amplification approach to create DNA templates on the bead for sequencing. The objective is to use neutral beads with a single tether and with forward and reverse primers for bridge PCR to generate the clonal copies on the bead.

Carboxylated polystyrene microbeads (r=0.5 μm, Bangs Laboratory) are functionalized using carbodiimide chemistry (EDC) by reacting with groups containing amines. Rather than using bridge PCR, generation of the beads suitable for this study can be simplified by attaching to the bead: (1) a low stoichiometric ratio of a tether dsDNA such that Poisson statistics favors one or zero tether per bead; (2) an amine-labeled primer hybridized to a 100 nt template oligonucleotide; and (3) excess ethanolamine to neutralize remaining unreacted carboxylic acids on the beads. A schematic of the bead functionalization is shown in FIG. 3(B).

Reaction conditions are provided to yield the desired number of templates per bead (100-5000). The template-to-bead ratios are determined by measuring the fluorescence of hybridized fluorescein labeled probe oligonucleotides and using a hemocytometer to measure the bead concentration. The dsDNA tether is constructed of DNA using amine and biotin labeled nucleotides. (See: Zimmermann, R. M. and E. C. Cox. “DNA stretching on functionalized gold surfaces.” Nucleic Acids Res., 1994, 22:492-7). Alternatively, the length of the tether DNA can be modified, as appropriate. The beads with a single tether molecule are captured on a surface by binding to adsorbed streptavidin. The number of non-DNA surface charges is quantified using the Zeta potential measurements, previously described. Beads of varying sizes (0.5-5 μm radius) are made to test in the optical measurements. Obtaining the correct stoichiometry of functional groups on the bead surface (tether, templates and non-DNA charge) is a key goal.

The bead preparation method is utilizes well-tested and understood surface attachment chemistries. An alternative method, if desired, is to use lipid coated beads as described by McLaughlin (See: McLaughlin S. “The electrostatic properties of membranes.” Annu. Rev. Biophys. Biophys. Chem., 1989, 18:113-36) to form neutral beads and attach templates and the tether with cholesterol labeled oligonucleotides. If the stretched tethers bring the beads too close to the substrate surface, streptavidin-labeled beads can be bound to the surface and used to capture the tethered beads. In this way, a spacer is placed between the substrate surface and the tethered bead to reduce or avoid hydrodynamic interactions between the bead and the surface. Alternatively, paramagnetic beads with magnetic force can be utilized as the restoring force. (See: Gosse, C. and V. Croquette. “Magnetic Tweezers: Micromanipulation and Force Measurement at the Molecular Level.” Biophys. J., 2002, 82:3314-29). While the use of magnetic fields to confine the beads may involve some complexities in the optical setup, it offers an alternative for the generation of large bead arrays.

Zeta Potential Measurement Using a Quadrant Photodiode on a Single Bead with Attached DNA

With reference to FIG. 9, a detection and measurement system is constructed that includes a quadrant photodiode, indicated at 50, to measure the Zeta potential of a tethered microbead similar to the one successfully used by Galneder et al. (See: Galneder, R., V. Kahl, A. Arbuzova, M. Rebecchi, J. O. Rädler and S. McLaughlin. “Microelectrophoresis of a bilayer-coated silica bead in an optical trap: application to enzymology.” Biophys J., 2001, 80:2298-309). A Nikon Diaphot 300 microscope, mounted on an optical table, is used to image a tethered bead (not shown) in a bead chamber, at 55, onto a Hamamatsu quadrant diode S5981, at 50 (Hamamatsu, Japan) (see FIG. 9). An objective, 52, is varied (2.5× to 40×) so as to match the bead size with the detector size. Illumination is with red-filtered light from a mercury arc lamp, 62 (via a mirror 58 and a condenser 56), using dark-field illumination. The quadrant photodiode circuit is as previously described (See: Simmons, R. M., J. T. Finer, S. Chu S and J. A. Spudich. “Quantitative measurements of force and displacement using an optical trap.” Biophys J., 1996, 70:1813-22). The amplified signal from the photodiode is captured with a DaqCard 1200 at 10 kHz using LabVIEW software. Raw data is converted to Zeta potential as previously described (See: Galneder, R., V. Kahl, A. Arbuzova, M. Rebecchi, J. O. Rädler and S. McLaughlin. “Microelectrophoresis of a bilayer-coated silica bead in an optical trap: application to enzymology.” Biophys J., 2001, 80:2298-309).

To measure the electrical charge on the bead, an AC voltage is applied (to induce periodic microelectrophoresis due to the charge of the attached DNA) with a DC bias (to stretch the DNA tether in order to tune the magnitude of the entropic force) at a frequency ranging from 10-200 Hz using a function generator (DS335, Stanford Research Systems) coupled to a high voltage amplifier (AS-1B3, Matsusada). Four sets of beads are prepared with known number of nucleotides by hybridizing DNA of lengths, 20, 50, and 51, and 100 bp to the templates on the beads to evaluate the sensitivity, dynamic range, and resolution of the measurements. Experimental parameters including electric field strength (AC and DC components), signal averaging time, and field frequency are selected so as to provide conditions for single base resolution at long read lengths. AC field in the range from 20 to 1000 V/cm is investigated.

Sequencing on a Bead Up to 75 nt

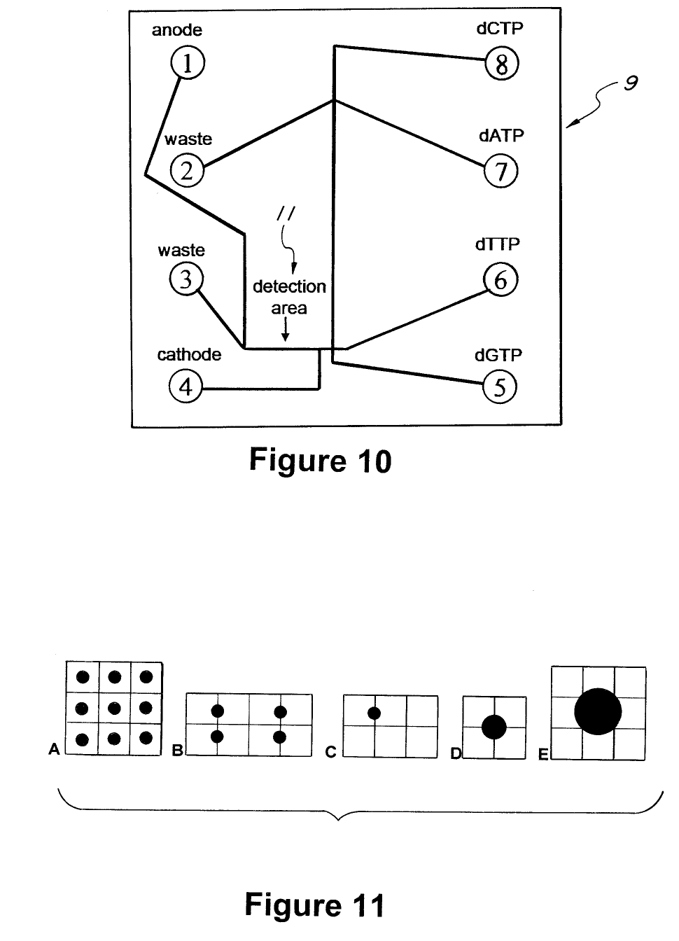

A chip, such as chip 9 depicted schematically in FIG. 10, is employed as the flow cell. The chip 9 (e.g., AMS365, Caliper Life Sciences) includes a fluidic network with 4 separate reservoirs, denoted as 5, 6, 7, and 8, for introducing the 4 dNTP's independently into the detection, at 11, area containing the captured beads. Chip 9 also includes anode and cathode buffer wells, 1 and 4, respectively; and two waste wells, 2 and 3. An Agilent Bioanalyzer power supply is used to sequentially drive electrophoresis of the dNTPs and polymerase over the bead. In the sequencing experiment, the microbeads (described above under the heading, “An entropic opposing force via tethering individual microbeads onto a glass substrate with dsDNA”) with clonal DNA attachment are tethered onto a detection area of the flow cell. The 4 dNTP's are introduced sequentially to the cell along with polymerase via electrophoresis. In the presence of appropriate conditions, including both an appropriate base and a polymerase, one nucleotide at a time is synthesized along the attached templates on the bead, and the increased bead charge is measured using the apparatus and method described above under the heading, “Zeta potential measurement using a quadrant photodiode on a single bead with attached DNA.” Bases are called when the signal changes by more than two times the background noise. The number of repeats in a homopolymeric region is determined by dividing the change in signal amplitude by the average change in amplitude for a one nucleotide addition. The read length and accuracy are optimized by varying the number of oligos per bead, the size of the bead, the electric field strength, the field frequency and observation time.

Part B: Optical Detection System Scalable to a Large Bead Array

This aspect of the present illustrative example relates to transitioning from a single detector to an imaging detector capable of measuring multiple beads. Initially, the detector is used with a single bead, then it is used with bead arrays.

Imaging System for Rapid Image Capture

This example provides an illustrative demonstration of systems and methods to measure, in parallel, the Zeta potential of a large number of beads. An optical system including an imaging detector is employed to determine the number of pixels that generate a suitable signal. The Nikon Diaphot 300 microscope with dark-field illumination is coupled to an M3 megapixel CMOS camera (IDT) capable of acquiring 1280×1024 images at 500 frames per second or higher frame rates at lower resolution. While it is expected that lower frame rates can be used in sequencing experiments of this example, the higher frame rate of this camera can be used to define the dependence of overall signal-to-noise on the electric field frequency and the camera frame rate.

As described above under the heading, “An entropic opposing force via tethering individual microbeads onto a glass substrate with dsDNA,” beads are bound with ˜1,000 templates/bead and linked to a surface via a biotin-labeled X, DNA. Single beads are imaged with 2.5×, 0.1 NA; 10×, 0.2 NA; 20×, 0.4 NA; and, 40×, 0.7 NA objectives. As shown in FIG. 11, different schemes including different numbers of pixels per bead are evaluated for quantifying the bead position. For the two pixel configuration (FIG. 11B), the image of the bead (r=0.5 μm) is placed at the midpoint of the edge of two pixels with the electric field perpendicular to the edge of the two pixels. For a sensor with a pixel size of 12 μm, it is expected to be possible to confine the image of the beads to two pixels with the 2.5× objective. Alternatively, buffer pixels can be used to prevent crosstalk between beads (FIG. 12C).

In alternate configurations, the bead image can be placed at the corner of a pixel (FIG. 12D) or the center of the pixel (FIG. 12E) to use 4 and 9 pixels per bead respectively. Magnification (and resolution) is varied by choice of objectives to maximize the signal-to-noise ratio.

Image Processing Algorithm to Convert Raw Images to Power Spectra

A program is written in C for calculating from the CMOS sensor images the positional information (analogous to the information processing carried out electronically by a quadrant photodetector circuit). The initial position of the bead is found by locating the two pixels in a user-defined sub-array which are above a user-defined threshold. For each image frame, the difference in the intensity of the two pixels is calculated and recorded. Fast Fourier transforms of the resulting time series are computed and the corresponding power spectra generated. The amplitude of the power spectral density at the electric field oscillation frequency is measured relative to the background. The Zeta potential is proportional to the square root of the amplitude.

Raw images are analyzed using ImageJ to quantify the amount of spread of the imaged bead onto adjacent pixels (if any). As described above under the heading, “Imaging system for rapid image capture,” confining the image in the pixels in line with the edge containing the bead image can be complex, so a weak cylindrical lens can be placed in the optical path so that the image is compressed in that dimension if there is leakage beyond the required number of pixels. The system, as described thus far, is then used to define the operating conditions for the illustrative a part of this example, next described.

Sequencing from Single Beads to 75 nt with >90% Accuracy Using CMOS Detector

Beads with bound templates are generated and attached to the surface of the flow cell, as described above under the heading, “An entropic opposing force via tethering individual microbeads onto a glass substrate with dsDNA.” As described above under the heading, “Sequencing on a bead up to 75 nt,” images are acquired in the presence of an electric field after each addition of dNTP. As described above under the heading, “Imaging system for rapid image capture,” regions with individual beads are selected and analyzed. The square root of the spectral power density is plotted as a function of added dNTP. Bases are called as described above under the heading, “Sequencing on a bead up to 75 nt.” The amplitude of the AC and DC components of the field, the electric field oscillation frequency, illumination intensity and the data acquisition frequency (by sampling the 1 kHz data) are varied to ascertain optimal sequencing conditions yielding sequences from individual beads to 75 nt with >90% accuracy. System performance (minimum detectable Zeta potential change, number of pixels between beads, sampling rate) parameters are extracted from the data and used with the model of predicted behavior towards configuring a system for generating reads at a rate of 2 GB/day.

Part C: Various Considerations and Alternative Strategies

Opposed-Forces Sequencing Using a Polymer Tether

In carrying out this aspect of the present illustrative example, care is taken to: (1) generate beads having the appropriate functional groups, (2) configure a polymer tether to providing a restoring force, and (3) optimizing the Zeta potential measurement to achieve suitable signal-to-noise-ratio. As described above, in the systems and apparatus of this example, beads are preferably near neutral before addition of template DNA in order to maximize sensitivity. Alternatively, optimized, lipid coated beads with intercalated cholesterol-labeled DNA can be used. While the magnitude of the DC field applied to the beads is expected to provide for some tuning of the spring constant of the DNA tether, other polymers such as dextran can be utilized as the tether. Additionally, tethers such as dextran contribute relatively little charge to the bead thereby increasing sensitivity. Attaining an acceptable bead:tether:surface stoichiometry, in some circumstances, may be challenging for tethers, such as dextran, which do not have clear attachment points limited to chain termini. In another approach, short synthetic polypeptides are polymerized to generate a tether with an acceptable spring constant, charge and attachment points. Care is taken in the optimization of the Zeta potential measurement, as it can be impact sensitivity for sequencing. Parameters such as the tether spring constant, electric field frequency, detector bandwidth and bead parameters are tested.

Optical Detection System Scalable to a Large Bead Array

In carrying out this aspect of the present illustrative example, care is taken to: 1) use a low number of pixels per bead, and 2) confine the bead image to prevent inter-pixel cross-talk. The strategies described above can be tested to define the minimum pixel number. Bead radius can be varied to optimize both the signal intensity changes in a pixel and the amount of spread of the bead image to adjacent pixels. As described in previous sections, cylindrical lenses can be introduced into the optical path to help limit the spread of the bead image. As an alternative to the proposed analog of the quadrant photodetector, structured illumination can be used to convert bead displacement to intensity fluctuations.

### Example 3

### Single Base Detection Sensitivity

This example is to illustrate sensitivity to single-nucleotide changes using opposing-forces sequencing, in accordance with the teachings herein.

Sensitivity with Individual Beads—without Using a Restoring Force

Single bead sequencing experiments useful to ascertain reliability of detection of single nucleotide incorporation events using opposing-forces sequencing.

Avidin beads labeled with biotin primed template oligonucleotides, and suspended in polymerization buffer (10 mM Tris, pH 8.1, 50 mM KCl, 50 mM TAPS, and 1% polymethylmethacrylate gel to suppress electroosmotic flow), are provided in a microfluidic chip. Bead velocity is measured from images from a CCD camera attached to a microscope (brightfield). The system electrokinetically transports the beads along a main channel and past a side channel configured to add a specific dNTP at 10 μM. Incubation proceeds for 1-minute in the presence of Klenow, exo-polymerase, and electrophoresis is performed to separate free dNTPs and the bead velocity is re-measured. The motions of an individual bead are imaged after each dNTP addition from subsequent side channels. Without a tether-based restoring force, the chip design limits the number of dNTP additions to an individual bead.

A change in relative bead velocity for sequential dNTP addition to beads with a primed template, in accordance with modeling and calculations, is expected to show a pattern; namely, statistically significant changes in velocity following addition of the correct nucleotide and no change in the velocity after addition of the incorrect nucleotide. Additionally, the change in signal is expected to exhibit proportionality to the number of added nucleotides.

Sensitivity with Individual Beads: Using Tethered Beads as Means to Provide a Restoring Force

In a modification of the experiments just discussed, tethered, optimized beads are used. Longer read lengths and increased accuracy, as compared to a system lacking such a restoring force, are expected.

These experiments are illustrative for ascertaining nucleotide incorporation for sequencing-by-synthesis on an individual bead, as far down as to single nucleotide sensitivity.

Sensitivity Enhancement

Here, methods are set forth to enhance precision of velocity measurement and, thus, increase attainable read length. It is noted that with the beads in the above experiments of this example, more than ⅔ of the charges originate from the beads themselves, rather than the oligonucleotides. One enhancement, aimed at the issue just mentioned, contemplates use of an approach utilizing covalent attachment of oligonucleotides to the beads to better optimize the surface charge density, such that less than ⅓ of the charges originate from the beads themselves. Another enhancement contemplated the use of tethered beads that experience a restoring force, in addition to other forces, such as the electrical and hydrodynamic forces in the initial experiments of this example, above. The restoring force is aimed at reducing noise otherwise introduced by movement of the beads due to Brownian motions while allowing larger electric fields to be used to maximize the velocity signal. Together, this allows more precise velocity measurements with higher signal-to-noise. Refinements, such as these, are each expected to lead to >2-fold improvement in the sensitivity of bead charge measurement and, thus, to single base resolution beyond a 50-base read length.

Summary

In this example, model systems and methods are set forth that include various features for sequence analysis using opposing forces, as taught herein. The experiments set forth are illustrative to ascertain sensitivity to acquire single base resolution (e.g., with a 10-base read length) using single-bead sequencing-by-synthesis. Predicted signal-to-noise ratio enhancement, provided for example by tethering optimized beads, is expected to provide for sequencing a 50-mer with single base sensitivity.

### Example 4

### Introduction

This example provides an illustrative demonstration of systems and methods of sequence determination by use of opposing forces; and, is particularly illustrative with regard to single-molecule sequencing (also referred to as “SMS” or “third-generation” sequencing) carried out on one bead.

As will be seen, a number of items are addressed in connection with this illustrative example, including: (1) making neutral gold beads with <1,000 charges/bead with single attached template DNA; (2) attaching beads to a surface with a single polymer tether (>90% with a single tether); (3) measuring single bead velocity using a quadrant photodiode system with sensitivity <1%; (4) detecting single charge differences for 50, 100, 150 and 200 nt templates; and, (5) sequencing on individual beads to 25 nt with 90% accuracy.

Part A: High Light-Scattering Cross-Section Beads with Single DNA Templates

To maximize sensitivity, it is desirable to: (1) keep the charge of the beads near neutral; (2) use small beads; (3) use no more than one tether per bead to control the stiffness of the entropic spring; and, (4) control the bead surface chemistry to prevent reagent sticking. Some or all of these are addressed, for example, by: (1) employing amino-PEG to block surface charges; (2) employing monodisperse gold beads because of their large light-scattering cross-section (See, e.g., Yguerabide, J. and E. E. Yguerabide. “Light-scattering submicroscopic particles as highly fluorescent analogs and their use as tracer labels in clinical and biological applications.” Anal. Biochem., 1998, 262:137-56), and availability in diameters <250 nm; (3) employing PEG as the tether chain as it is uncharged and available with well-defined heterobifunctional ends; and, (4) taking advantage of the amino-PEG coating to minimize non-specific binding.

Carboxy-functionalized gold microbeads (r=0.05 μm, Nanopartz) are functionalized using carbodiimide chemistry (EDC) by reacting with groups containing amines: (1) a low stoichiometric ratio of a biotin PEG-amine (IRIS Biotech) such that Poisson statistics favors one or zero tether per bead; (2) an amine-labeled DNA primer; and (3) excess amino-PEG to neutralize remaining unreacted carboxylic acids on the beads. Reaction conditions are optimized to yield on average ˜5 to 100 primers per bead. Templates of desired length are annealed to the capture primers to give on average one primed template per bead. The template-to-bead ratio is determined by measuring the fluorescence of hybridized fluorescein-labeled probe oligonucleotides and using a hemocytometer to measure the bead concentration. The length of the tether can be modified by varying the PEG molecular weight (typically 10,000 to 100,000 Da). The beads with a single tether molecule are captured on a surface by binding to adsorbed streptavidin. The number of bead surface charges is quantified using the velocity measurements, as previously described. Reaction conditions are optimized to obtain the correct stoichiometry of functional groups on the bead surface (tether, templates and non-DNA charge). It is anticipated that the PEG coating will prevent aggregation of the beads (See, e.g., Lasic, D. D., F. J. Martin, A. Gabizon, S. K. Huang, D. Papahadjopoulos. “Sterically stabilized liposomes: a hypothesis on the molecular origin of the extended circulation times.” Biochim Biophys. Acta. 1991, 1070:187-92).

Part B: Single Base Resolution Sequencing on Single DNA Molecules.

A planar chip (e.g., AMS365; Caliper Life Sciences) is used as a flow cell. The chip includes a fluidic network with four separate reservoirs for introducing the four dNTP's independently into the detection area containing the captured beads (See: Chow, A. W. “DNA separations.” Methods Mol. Biol., 2006, 339:129-44). An Agilent Bioanalyzer power supply is used to sequentially drive electrophoresis of the dNTPs and polymerase over the bead. In an illustrative sequencing experiment, microbeads (prepared as described above) are tethered onto the detection area of the flow cell by binding of the biotinylated PEG tether to surface adsorbed streptavidin. The four dNTP's are introduced sequentially to the bead. In the presence of the appropriate nucleotide (a complementary nucleotide), polymerization along the template is detected as a change in the amplitude of bead motion in the oscillating electric field. Bases are called when the signal changes by more than two times the background noise. The number of repeats in a homopolymeric region is determined by dividing the change in signal amplitude by the average change in amplitude for a one nucleotide addition. Calculations indicate that the change in signal amplitude is linearly proportional to the number of charges over tens of nucleotides. The read length and accuracy are optimized by varying the size of the bead, the electric field strength, the field frequency, observation time, and buffer composition.

Part C: Various Considerations and Alternative Strategies

High light-scattering cross-section beads with single DNA templates

Care is taken to generate beads with the appropriate functional groups. The illustrative bead preparation method, here, is based on well-tested and understood surface attachment chemistries. If desired, an alternative method involves using lipid-coated beads, as described by McLaughlin (See: Galneder, R., V. Kahl, A. Arbuzova, M. Rebecchi, J. O. Rädler and S. McLaughlin “Microelectrophoresis of a bilayer-coated silica bead in an optical trap: application to enzymology.” Biophys J., 2001, 80:2298-309), to form neutral beads and attach templates and the tether via a cholesterol label.

Single Base Resolution Sequencing on Single DNA Molecules

Care is taken to achieve suitable signal-to-noise-ratio to detect single nucleotide charge changes.

In another approach, ligation is used instead of polymerization (See: Shendure, J. et al. “Accurate multiplex polony sequencing of an evolved bacterial genome.” Science, 2005, 309:1728-1732). The template DNA is sequentially probed with 7-mer oligos (NNNXNNN, where X is one of the four dNTPs). This is expected to yield a charge difference seven times greater than that for incorporation of a single nucleotide by polymerization.

While the principles of the present teachings have been illustrated in relation to various exemplary embodiments shown and described herein, the principles of the present teachings are not limited thereto and include any modifications, alternatives, variations and/or equivalents thereof. All such modifications, alternatives, and equivalents are intended to be encompassed herein.

This application incorporates by reference in their entirety for all purposes all publications, patents, and patent applications cited herein.

It is claimed:

1. A system for determining sequence information of a polymeric analyte, wherein the analyte includes (i) at least one nucleic-acid molecule comprising a number of monomer units and (ii) a quantity of charge, with the quantity being a function of the number; said system comprising:
first and second sources of force, each configured to apply, when active, a respective force that acts upon said analyte; and wherein the applied forces include vector components in opposing relation; and further wherein the forces include an electrical force, strongly dependent on the quantity of charge of said analyte, and at least one additional force, weakly or non-dependent on the quantity of charge of said analyte;
a reagent region, and a composition of matter therein; wherein said composition of matter comprises a mixture being characterized by an enzyme activity effective to change the number of monomer units, when in effective proximity with the analyte;
a support element comprising a microbead, characterized by a detectable parameter, disposed in close association with the analyte such that a change in the number of monomer units, accompanied by a change in the charge, effects a change in the detectable parameter;
a surface and a tether, with the tether comprising a biological polymer; wherein the tether extends between the surface and the microbead in a manner to tether the microbead to said surface; and wherein said tether can provide a spring-like, restoring force;
a pathway leading the composition into effective proximity with the analyte;
a detector configured to detect a change in the detectable parameter of the support element; and,
a processing unit, adapted to receive signal information from the detector, and programmed to process the information and provide sequence information relating to one or more monomer units of the polymeric analyte.

2.-6. (canceled)

7. A method for analyzing sequence information of a polymeric analyte, with said analyte including (a) at least one nucleic-acid molecule comprising a DNA molecule, and (b) a number of monomer units and a net charge which is a function of the number; the method comprising:
(i) supporting the analyte with a particle; wherein the particle includes a detectable parameter that is a function of the charge;
(ii) forming an array comprising the analyte-supporting particle and a plurality of additional analyte-supporting particles;
(iii) tethering the particles of the array to a surface with respective tethers which, at least in part, can provide a restoring force;
(iv) applying opposing forces so as to act upon the array of analyte-supporting particles; wherein one of the forces is characterized by a dependence on the charge of the analyte;
(v) detecting and measuring the parameter;
(vi) changing the number of monomer units of the analyte; wherein said changing is carried out by synthesizing nucleic acid, cleaving nucleic acid, binding an oligonucleotide, displacing a bound oligonucleotide, or ligating nucleic acids;
(vii) detecting and measuring for a consequential change in the parameter; and
(viii) analyzing any changed parameter, thereby determining sequence information relating to the analyte;
wherein a plurality of the steps of said method are carried out substantially simultaneously on said array of particles; and further wherein a plurality of contiguous nucleic acids of DNA are sequenced so as to provide single-base resolution sequencing.

8.-20. (canceled)

21. A method for sequencing one or more nucleic-acid polymers, comprising:
(i) generating a plurality of analyte complexes, with individual complexes of the plurality including (a) a bead support, at least one nucleic-acid polymer, and a quantity of associated nucleic acids, wherein individual nucleic acids of the quantity are unlabeled and include a charge, (b) a net charge that depends on the quantity of associated nucleic acids, and (c) a kinematic property that depends on the net charge;
(ii) forming an analyte array comprising the plurality of analyte complexes;
(iii) acting upon the analyte array with a plurality of forces, with the forces including (a) an electrical force and a restoring force, and (b) vector components disposed in opposing directions;
(iv) imaging the analyte array to detect for signals corresponding to the kinematic property, to obtain a first set of measurements;
(v) subjecting the analyte array to reagents and conditions suitable for a sequencing-by-synthesis reaction, whereby the quantity of associated nucleic acids of one or more analyte complexes of the analyte array can be changed in a sequence-dependent manner;
(vi) imaging the analyte array to detect for signals corresponding to the kinematic property, to obtain a second set of measurements;
(vii) comparing first and second sets of measurements to identify differences indicating one or more sequencing-by-synthesis incorporation events; and
(viii) repeating steps (iii) through (vii);
whereby at least one or more portions of said at least one nucleic-acid polymer of the analyte complexes are sequenced.

22. The method of claim 21, wherein said at least one nucleic-acid polymer is DNA or RNA; wherein said imaging steps are effected at least in part using an imaging detector; and further wherein said restoring force is provided at least in part by a tether or a dielectrophoretic trap.

23. The method of claim 21, wherein the plurality of forces, recited in step (iii), act upon the analyte array at least during both of the imaging steps, recited in steps (iv) and (vi).

24. A system for sequencing nucleic-acid polymers, comprising:
means providing a plurality of analysis-sites for receiving a plurality of complexes for analysis in parallel, with each complex including, in combination, one or more nucleic-acid polymers, a means for supporting said one or more nucleic-acid polymers and a quantity of associated charged nucleic acids; wherein the complex is characterized by a net charge dependent on the quantity of associated charged nucleic acids, and a kinematic property dependent on the net charge;
means for applying plural forces for simultaneously acting at the plurality of analysis sites, wherein the forces include vector components disposed in opposing directions, and at least one the forces includes a dependence on the net charge;
means capable of changing the quantity of associated charged nucleic acids by sequence-dependent addition of nucleic-acid monomers;
means for measuring the kinematic property at (i) a time before and (ii) a time at or after an action of the means capable of changing;
means for comparing measurements to determine a difference; and
means for processing results generated by the means for comparing, to thereby determine one or more sequences.

25. A system for analysis of at least one user-provided analyte complex, which complex includes a biological-polymer comprising a sequence of monomers, an associated support and a quantity of associated charged moieties; wherein the complex includes a net charge that depends on the quantity of associated charged moieties, and a kinematic property that depends on the net charge; the system comprising:
an analysis site adapted to receive the analyte complex;
means for applying opposing forces at the analysis site, with at least one of the forces including a strong dependence on the net charge, and another of the forces including no more than a weak dependence on the net charge;
means capable of changing the quantity of associated charged moieties, with such capability including a dependence on a given sequence;
means for detecting the kinematic property at (i) a time before and (ii) a time at or after action of the means capable of changing;
means for measuring detected kinematic properties;
means for comparing measurements of detected kinematic properties; and
means for analyzing results relating to detected kinematic properties, and determining sequence information.

26. The system of claim 25, further comprising:
means for determining a difference between measurements of detected kinematic properties; and
means for quantifying the difference in the event the determined difference is non-zero.

27. The system of claim 25, wherein a CMOS device comprises, at least in part, two or more of said means for detecting, means for measuring, means for comparing, means for analyzing, means for determining a difference, and means for quantifying.

28. The system of claim 25, wherein said means for applying opposing forces comprises (i) a source for providing an electrical force that is dependent on the net charge, and (ii) a means for providing a restoring force, which exhibits little to no dependence on the net charge.

29. A method for analyzing a sequence characteristic of at least one polymeric molecule, the method comprising:
(i) forming an analyte complex that includes (a) at least one polymeric-molecule analyte, a support therefore and a quantity of associated charged moieties, (b) a net charge that depends on the quantity of associated charged moieties, and (c) a kinematic property that depends on the net charge;
(ii) acting upon the analyte complex with plural forces, which include vector components disposed in opposing directions, with at least one of the forces being dependent on the net charge of the analyte complex;
(iii) measuring the kinematic property of the analyte complex;
(iv) next, subjecting the analyte complex to an agent or event capable of changing the quantity of charged moieties associated with the polymeric molecule, with such capability being dependent on the sequence characteristic;
(v) then, again measuring the kinematic property of the analyte complex; and
(vi) comparing measurements from steps (iii) and (v) to determine a difference, thereby obtaining information relating to the sequence characteristic.

30. The method of claim 29, further comprising: repeating steps (ii) through (vi) one or more times, thereby obtaining additional information for one or more monomer units comprising said at least one polymeric-molecule analyte.

31. The method of claim 30, wherein said at least one polymeric-molecule analyte comprises a biological polymer; and wherein the system further comprises the step of processing the information so as to define a sequence of contiguous monomer units.

32. The method of claim 31, wherein said method is carried out on at least 1,000 analyte complexes in parallel, with said at least 1,000 analyte complexes being simultaneously imaged.

33. The method of claim 31, wherein the biological polymer comprises a nucleic-acid polymer, and the charged moieties comprise nucleic acids.

34. The method of claim 33, wherein said analyte complex includes no more than a single nucleic-acid polymer.

35. The method of claim 33, wherein said analyte complex includes a plurality of nucleic-acid polymers, wherein said plurality of nucleic-acid polymers comprises replicates of a nucleic-acid polymer of interest.

36. The method of claim 35, wherein step (i) comprises forming a plurality of distinct, unique analyte complexes.

37. The method of claim 33, wherein said support of the analyte complex comprises a particle.

38. The method of claim 37, wherein said kinematic property is position, linear velocity, rotational velocity, acceleration, or a combination of the foregoing.

39. The method of claim 37, wherein plural analyte complexes are analyzed in parallel, with each including a particle supporting one or more associated nucleic-acid polymers; and wherein at least 1,000 analyte complexes are analyzed in parallel; and further wherein said at least 1,000 analyte complexes are configured in an array.

40. The method of claim 39, further wherein said measuring steps include imaging said array.

41. The method of claim 31, wherein said plural forces include an electrical force, which electrical force comprises, at least in part, said at least one of the forces that is dependent on the net charge.

42. The method of claim 41, wherein the method steps are carried out on at least 1,000 analyte complexes in parallel; and wherein the method further comprises the step of configuring said at least 1,000 analyte complexes so as to define an array.

43. The method of claim 41, wherein said support of said analyte complex comprises a particle; and further wherein one or more of said plural forces comprises a restoring force.

44. The method of claim 43, wherein said restoring force is a tethering force, an optical trapping force, a magnetic trapping force, a dielectrophoretic trapping force, or a combination thereof.

45. The method of claim 44, wherein said restoring force comprises a tethering force, provided at least in part by a polymeric molecule tethering said analyte complex.

46. The method of claim 33, wherein said support comprises a particle; and wherein said nucleic-acid polymer comprises at least one polynucleotide; and further wherein step (iv) includes changing the amount of nucleic acids associated with the at least one polynucleotide.

47. The method of claim 46, wherein said changing the amount of nucleic acids associated with the at least one polynucleotide includes providing one or more primed polynucleotides, and sequentially subjecting said one or more primed polynucleotides to reagents and conditions suitable for sequencing-by-synthesis reactions.

48. The method of claim 46, wherein said changing the amount of nucleic acids is effected by one of: (a) synthesizing one or more nucleic acids to the polynucleotide bound to the particle, (b) binding one or more oligonucleotides to the polynucleotide bound to the particle, (c) displacing one or more bound oligonucleotides from the polynucleotide bound to the particle, or (d) ligating one or more nucleic acids to the polynucleotide bound to the particle.

49. A system for analyzing a sequence characteristic of at least one polymeric molecule; the system for use with an analyte complex that includes (a) at least one polymeric-molecule analyte, a support therefore and a quantity of associated charged moieties, (b) a net charge that depends on the quantity of associated charged moieties, and (c) a kinematic property that depends on the net charge; the system further for use with at least one analytic reagent mixture capable of changing the quantity of associated charged moieties, with such capability being dependent on the sequence characteristic; the system comprising:
a reaction region, including an analysis site adapted to receive and support the analyte complex, when provided for analysis;
first and second sources of force, each configured to apply, when in an activated state, a respective force along the reaction region, with the applied forces (i) arranged to act at the analysis site, and (ii) including vector components disposed in opposing directions;
a reagent source, an entry to the reaction region, and a reagent-delivery path extending from the reagent source, through the entry, and to the analysis site; wherein the path is configured to facilitate contact between an analytic reagent mixture, when provided by the source for delivery along the path to the analysis site, and the analyte complex;
a detector operatively configured (i) to observe at least a portion of the reaction region, including the analysis site, while the sources of force are in the activated state and the applied forces present, and (ii) to detect for first and second signals, separated by a temporal interval, relating to the kinematic property of the analyte complex; with the temporal interval comprising a duration sufficient to allow delivery of the analytic reagent mixture to the analysis site; and
a signal processor adapted to receive detected first and second signals, and configured to compare the signals and determine a difference, thereby obtaining information relating to the sequence characteristic.

50. The system of claim 49, further comprising one or more additional sources of force configured to apply, when in an activated state, one or more respective forces along the reaction region.

51. The system of claim 50, wherein said sources of force include one or more sources configured for providing an electrical force and a restoring force.

52. The system of claim 49, further comprising a flow cell, with said flow cell including: (i) a chamber defining said reaction region, (ii) at least one reservoir defining said reagent source, and, (iii) one or more channels between said one or more reservoirs and said chamber.

53. The system of claim 49, wherein said detector comprises an imaging array detector which includes an array of pixels.

54. The system of claim 53, wherein said detector comprises a CMOS sensor and a logic device programmed for calculating kinetic-parameter information from a plurality of sensor images.

55. The system of claim 49, further comprising a processing means programmed to interpret detector signal information and provide output corresponding to sequences of nucleic acids.
